(19) United States
(12) Patent Application Publication
(10) Pub. No.: US 2020/0237789 A1
(43) Pub. Date: Jul. 30, 2020

(54) COMBINATION THERAPY FOR PREVENTING ADDICTION

(71) Applicant: Amygdala Neurosciences, Inc., Palo Alto, CA (US)

(72) Inventors: Brent Blackburn, Los Altos, CA (US); Ivan Diamond, Berkeley, CA (US); Louis G. Lange, Palo Alto, CA (US); Peter M. Strumph, San Francisco, CA (US)

(73) Assignee: Amygdala Neurosciences, Inc., Palo Alto, CA (US)

(21) Appl. No.: 16/849,870

(22) Filed: Apr. 15, 2020

Related U.S. Application Data
(63) Continuation of application No. PCT/US2018/055937, filed on Oct. 15, 2018.
(60) Provisional application No. 62/643,612, filed on Mar. 15, 2018.
(60) Provisional application No. 62/573,062, filed on Oct. 16, 2017.

(51) Int. Cl. A61K 31/675; A61K 31/4418; A61P 25/30

(52) U.S. Cl. CPC A61K 31/675; A61P 25/30; A61K 31/4418

(57) ABSTRACT

Disclosed is a novel combination therapy to reduce or prevent the acquisition of a conditioned response in a mammal comprising administering to the mammal a therapeutically effective amount of an aldehyde dehydrogenase (ALDH-2) inhibitor compound, such as a compound of Formula (I), in combination with a substance that produces the conditioned response, such as a medication containing a dopamine-producing agent such as an opioid, whereby the combination acts to reduce or prevent the acquisition of a conditioned response, and the deleterious side-effect of misuse, dependence, abuse, and/or addiction.

### CROSS-REFERENCE TO RELATED APPLICATIONS

This application is a continuation of PCT Application No. PCT/US2018/055937, filed Oct. 15, 2018, which claims priority of U.S. Provisional Application No. 62/643,612, filed March 15, 2018 and 62/573,062, filed Oct. 16, 2017, the entireties of each of which are incorporated herein by reference.

### FIELD

The present disclosure relates to a novel combination therapy comprising administering to a mammal in need thereof an aldehyde dehydrogenase-2 (ALDH-2) inhibitor in combination with a substance that produces a conditioned response (e.g., a medication containing a dopamine-producing agent, such as an opioid medication), whereby the combination acts to reduce or prevent the side-effects of misuse, dependence, abuse, and/or addiction related to the substance. The disclosure further relates to methods and pharmaceutical compositions useful with the combination therapy.

### BACKGROUND

The United States Surgeon General has declared substance abuse a national health care crisis that is estimated to have resulted in greater than 3 months reduction in average U.S. life expectancy, 155,000 related deaths per year, 23 million needing treatment, and a $400 billion economic cost annually. See “Facing Addiction in America,” Surgeon General's Report, 2016. The Center for Disease Control estimates that illicit drug overdoses killed 64,000 people in the U.S. in 2016, with 14,000 of those deaths resulting from prescription opioid medications.

It has also been shown that inhibition of aldehyde dehydrogenase-2 (ALDH-2) can reduce pathophysiologic dopamine surge without changing basal dopamine levels in a rat model of cue-induced cocaine relapse-like behavior. See e.g., Yao et al., “Inhibition of aldehyde dehydrogenase-2 suppresses cocaine seeking by generating THP, a cocaine use-dependent inhibitor of dopamine synthesis,” Nature Medicine (2010), Vol. 16, No. 9; Diamond and Yao, “From Ancient Chinese Medicine to a Novel Approach to Treat Cocaine Addiction,” CNS & Neurological Disorders-Drug Targets (2015) Vol. 14, No. 6. Some studies have concluded that dopamine is essential for learning and performance of conditioned response (CR) behavior. See e.g., Darvas et al., “Dopamine dependency for acquisition and performance of Pavlovian conditioned response,” Proc. Natl. Acad. Sci. USA (2014), Vol. 111 (7): 2764-2769. A recent review concludes that dopamine surge above normal levels is part of the reward circuit common to all drugs of addiction. See e.g., Volkow et al., “Neurobiologic Advances from the Brain Disease Model of Addiction,” N. Engl. J. Med. (2016) 374:363-371. However, the issue of whether basal dopamine levels are sufficient to support acquisition of CR behavior, or if dopamine surges are required for acquisition is not known from recent studies of the physiology of addiction,

The isoflavone compound, daidzein, and several of its structurally related derivatives have been shown to be selective inhibitors of ALDH-2, relative to the MAO pathway, and exhibit effectiveness in treating alcohol dependency. See e.g., Keung et al., (1993) Proc. Natl. Acad. Sci. USA 90, 10008-10012; Keung et al., (1997) Proc. Natl. Acad. Sci. USA 94, 1675-1679; U.S. Pat. Nos. 5,624,910, 6,121,010, 7,951,813, 8,158,810, and 8,673,966; International Patent Publ. Nos. WO2008/014497, WO2008/124532, WO2009/061924, WO2009/094028, and WO2013/033377.

More recently, a genus of compounds, structurally unrelated to the isoflavones, with a core structure of Formula (I) (described below), such as 2,6-dichloro-N-[4-(2-oxo-1,2-dihydro-pyridin-4-yl)-benzyl]-benzamide (disclosed herein as compound (1)), have been shown to inhibit ALDH-2 selectively relative to the monoamine oxidase (MAO) pathway, and exhibit effectiveness in treating rat models of alcohol, nicotine, and cocaine dependency. See e.g., U.S. Pat. Nos. 8,558,001, 8,575,353, 9,000,015, 9,610,299; Int'l Pat. Publ. WO2013/006400; and Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6.

Numerous substances that produce a conditioned response in a mammal, such as medications that contain dopamine producing agents capable of inducing a dopamine surge, in particular opioid medications, remain medically necessary for the treatment of numerous pathologies (symptoms and diseases) in patients. For example, prescription of opioids to patients for post-surgical pain relief. Even the prescribed use of such medications, however, can result in the harmful side-effect in the patient of drug misuse, dependence, addiction, or other substance abuse disorders. Accordingly, there remains a need for pharmaceutical compositions and formulations of substances containing dopamine producing agents, particularly opioid medications, and associated methods of treatment, that reduce or prevent acquisition of a conditioned response and thereby prevent or decrease the potential side-effect of substance abuse disorders in a patient.

### SUMMARY

As described above, ALDH-2 inhibitors, such as compounds of Formula (I), have been shown to be effective against cue-induced relapse behavior in rat models of substance addiction. The present disclosure provides therapeutic methods that use these ALDH-2 inhibitors in combination with substances that produce a conditioned response, for example, medications that contain a dopamine-producing agent, such as opioid medications, to prevent individuals not previously addicted, from acquiring an addiction to the substance.

In some embodiments, the present disclosure provides a method of reducing or preventing addiction to a substance that produces a conditioned response in a mammal, the method comprising administering to the mammal a therapeutically effective amount of an ALDH-2 inhibitor in combination with the substance. In some embodiments, prior to administering the ALDH-2 inhibitor in combination with the substance the mammal has not acquired a conditioned response to the substance, the mammal does not have an addiction to the substance, and/or the mammal has not used, been treated with, or otherwise ingested the substance for a period of time of at least 1 month, at least 3 months, at least 6 months, at least 1 year, or ever. In various embodiments of the method, the ALDH-2 inhibitor and the substance are administered: (a) separately and not at the same time; (b) separately and at the same time; or (c) administered in a combination dosage form. In some embodiments of the method, the substance that produces a conditioned response is a medication, a medication, an extract, a food, alcohol, nicotine, amphetamine, or a drug of addiction. In some embodiments, the substance is a medication, optionally a medication comprising a dopamine-producing agent. In some embodiments, the substance is an opioid medication, optionally an opioid medication selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof. In some embodiments of the method, the mammal suffers from chronic pain and the medication is an opioid medication. In some embodiments of the method, the mammal has undergone a surgical procedure and the medication is a post-surgical treatment.

In some embodiments, the present disclosure provides a method of reducing or preventing addiction to a medication in a mammal, the method comprising administering to the mammal in need of the medication a therapeutically effective amount of an ALDH-2 inhibitor in combination with a therapeutically effective amount of the medication. In some embodiments, prior to administering the ALDH-2 inhibitor in combination with the medication the mammal has not acquired a conditioned response to the medication, the mammal does not have an addiction to the medication, and/or the mammal has not used, been treated with, or otherwise ingested the medication for a period of time of at least 1 month, at least 3 months, at least 6 months, at least 1 year, or ever. In various embodiments of the methods, the ALDH-2 inhibitor and the medication are administered: (a) separately and not at the same time; (b) separately and at the same time; or (c) administered in a combination dosage form. In some embodiments of the method, the medication that produces a conditioned response is a medication comprising a dopamine-producing agent, optionally, the mammal does not have an addiction to the dopamine-producing agent prior to the administering of the ALDH-2 inhibitor. In some embodiments, the medication is an opioid medication, optionally an opioid medication selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof. In some embodiments of the method, the mammal suffers from chronic pain and the medication is an opioid medication. In some embodiments of the method, the mammal has undergone a surgical procedure and the medication is a post-surgical treatment.

In some embodiments, the present disclosure provides a method of reducing or preventing in a mammal the acquisition of addiction to a medication that comprises a dopamine-producing agent, wherein the method comprises administering to the mammal a therapeutically effective amount of an ALDH-2 inhibitor in combination with the medication.

In some embodiments, the present disclosure provides a method of treating a mammal in need thereof with a medication that comprises a dopamine-producing agent, the method comprising administering to the mammal a therapeutically effective amount of the medication in combination with a therapeutically effective amount of an ALDH-2 inhibitor.

In some embodiments, the present disclosure provides a method of treating pain in a mammal in need thereof, comprising administering to the mammal a therapeutically effective amount of an opioid medication in combination with a therapeutically effective amount of an ALDH-2 inhibitor.

In some embodiments of the methods disclosed herein, the step of administering the medication containing the dopamine-producing agent in combination with the ALDH-2 inhibitor can comprise administering the medication and the ALDH-2 inhibitor separately. In some embodiments, the ALDH-2 inhibitor is administered as a once-a-day dose.

In various embodiments of the methods disclosed herein, the ALDH-2 inhibitor and the substance or medication are administered: (a) separately and not at the same time; (b) separately and at the same time; or (c) administered in a combination dosage form.

In some embodiments of the methods disclosed herein, the step of administering the substance or medication containing the dopamine-producing agent in combination with the ALDH-2 inhibitor can comprise administering a pharmaceutical composition, wherein the pharmaceutical composition comprises the substance or medication, the ALDH-2 inhibitor, and a pharmaceutically acceptable carrier. In some embodiments, the administered pharmaceutical composition is in a unit dosage form.

In some embodiments of the methods disclosed herein, the mammal is a human. In some embodiments, the mammal has undergone a surgical procedure and the substance or medication is a post-surgical treatment. In some embodiments, the mammal is suffering from chronic pain and the substance or medication is an opioid medication, optionally an opioid medication selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

In some embodiments of the methods disclosed herein, prior to the administering of the medication the mammal does not have an addiction to a dopamine-producing agent. In some embodiments, prior to the administering of the medication, the mammal has not been treated with, used, or otherwise ingested the medication for at least 1 month, at least 3 months, at least 6 months, at least 1 year, or ever.

In addition to the methods, in some embodiments the present disclosure provides a pharmaceutical composition comprising a therapeutically effective amount of an ALDH-2 inhibitor, a substance that produces a conditioned response in a mammal, and a pharmaceutically acceptable carrier. In some embodiments, the substance comprises a dopamine-producing agent. In some embodiments, the substance is a medication, an extract, a food, alcohol, nicotine, amphetamine, or a drug of addiction. In some embodiments, the substance is a medication, optionally an opioid medication selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

In some embodiments, the present disclosure also provides a combination pharmaceutical composition, wherein the composition comprises a therapeutically effective amount of a medication that comprises a dopamine-producing agent, a therapeutically effective amount of an ALDH-2 inhibitor, and a pharmaceutically acceptable carrier. In some embodiments, the pharmaceutical composition is in a unit dosage form.

In some embodiments of the pharmaceutical compositions disclosed herein, the substance, such as medication containing a dopamine-producing agent, is an opioid medication. In further embodiments, the opioid medication is selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

In some embodiments, the pharmaceutical composition disclosed herein are for use in therapy. In some embodiments, the disclosure provides for the use of a pharmaceutical composition for the manufacture of a medicament, wherein the medicament is for the treatment of a human in need thereof with a medication comprising a dopamine producing agent. In some embodiments, the use of the pharmaceutical composition is for the manufacture of a medicament for the treatment of pain in a human.

In some of the various embodiments of the methods and/or pharmaceutical compositions disclosed herein, the ALDH-2 inhibitor is a compound of Formula (I)

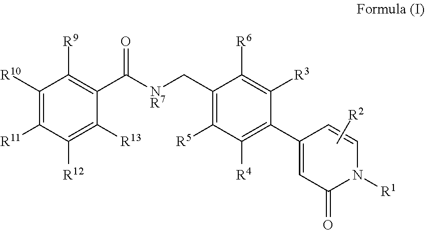

wherein:
R¹ is hydrogen, optionally substituted C₁₋₆ alkyl, —CH₂OH, —CH₂OP(O)(OR²⁰)(OR²¹);
R² is hydrogen, optionally substituted C₁₋₆ alkyl, cycloalkyl, or halo;
each of R³, R⁴, R⁵, R⁶, R⁹, R¹⁰, R¹¹, R¹² and R¹³ is independently hydrogen, hydroxyl, —OP(O)(O²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O-(C₁ to C₆-alkyl)-O-(C₁ to C₆-alkyl), cyano, halo, —SO₂NR²⁴R²⁵; or —NR²⁴R²⁵;
R⁷ is hydrogen or optionally substituted C₁₋₆ alkyl;
each of R²⁰ and R²¹ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and
each of R²⁴ and R²⁵ is independently chosen from hydrogen or C₁₋₆ alkyl or when combined together with the nitrogen to which they are attached form a heterocycle; or
a pharmaceutically acceptable salt, ester, single stereoisomer, mixture of stereoisomers, or a tautomer thereof.

In some embodiments of the methods and/or pharmaceutical compositions disclosed herein, the ALDH-2 inhibitor is a compound the compound of formula (I) is selected from the group consisting of: 2,6-dichloro-4-(2-methoxyethoxy)-N-(4-(2-oxo-1,2-dihydropyridin-4-yl) benzyl)benzamide; 2,6-dichloro-N-[4-(2-oxo-1,2-dihydro-pyridin-4-yl)-benzyl]-benzamide; 2-chloro-3-fluoro-N-(4-(2-oxo-1,2-dihydropyridin-4-yl)benzyl)benzamide; 2-chloro-6-methyl-N-(4-(2-oxo-1,2-dihydropyridin-4-yl)benzyl)benzamide; 2,6-dimethyl-N-(4-(2-oxo-1,2-dihydropyridin-4-yl)benzyl)benzamide; 2,6-dichloro-N-[4-(6-methyl-2-oxo-1,2-dihydro-pyridin-4-yl)-benzyl]-benzamide; 2-chloro-3,6-difluoro-N-(4-(2-oxo-1,2-dihydropyridin-4-yl)benzyl)benzamide; 2,6-dichloro-N-(3-methyl-4-(2-oxo-1,2-dihydropyridin-4-yl)benzyl)benzamide); 2,6-dichloro-N-(4-(1-methyl-2-oxo-1,2-dihydropyridin-4-yl)benzyl)benzamide; 2,6-difluoro-N-(4-(2-oxo-1,2-dihydropyridin-4-yl)benzyl)benzamide; 2-chloro-6-fluoro-N-(4-(2-oxo-1,2-dihydropyridin-4-yl)benzyl)benzamide; 2,6-dichloro-N-(2-fluoro-4-(2-oxo-1,2-dihydropyridin-4-yl)benzyl)benzamide; 2,6-dichloro-N-(4-(5-fluoro-2-oxo-1,2-dihydropyridin-4-yl)benzyl)benzamide; phosphoric acid mono-(4-[4-[(2,6-dichloro-benzoylamino)-methyl]-phenyl]-2-oxo-2H-pyridin-1-ylmethyl) ester; 2,6-dimethyl-N-(4-(2-oxopiperidin-4-yl)benzyl)benzamide; or a pharmaceutically acceptable salt, single stereoisomer, mixture of stereoisomers, or a tautomer thereof.

In some embodiments of the methods and/or pharmaceutical compositions disclosed herein, the ALDH-2 inhibitor is a compound of formula (I), wherein the compound of formula (I) is compound (1):

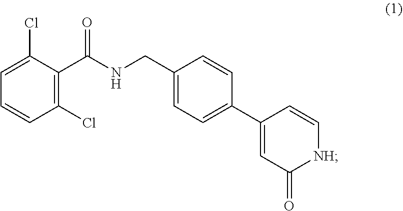 [SMILES: O=C(NCc1ccc(-c2cc[nH]c(=O)c2)cc1)c1c(Cl)cccc1Cl]

or a pharmaceutically acceptable salt, or a tautomer thereof.

In some embodiments of the methods and/or pharmaceutical compositions disclosed herein, the ALDH-2 inhibitor is a compound of formula (I), wherein the compound of formula (I) is compound (2):

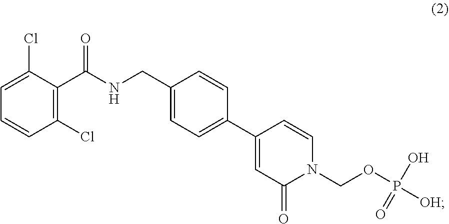 [SMILES: O=C(NCc1ccc(-c2ccn(COP(=O)(O)O)c(=O)c2)cc1)c1c(Cl)cccc1Cl]

or a pharmaceutically acceptable salt, ester, or a tautomer thereof.

In some embodiments of the methods and/or pharmaceutical compositions disclosed herein, the ALDH-2 inhibitor is a compound comprising an isoflavone structure. In some embodiments, the compound comprising an isoflavone structure is daidzein (compound (15)):

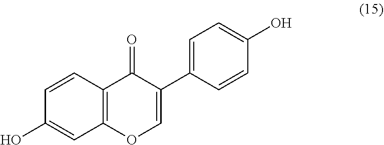 [SMILES: O=c1c(-c2ccc(O)cc2)coc2cc(O)ccc12]

or a pharmaceutically acceptable salt, ester, or a tautomer thereof. In some embodiments, the compound comprising an isoflavone structure is 3-{[3-(4-aminophenyl)-4-oxochromen-7-yloxy]methyl}benzoic acid (compound (16)):

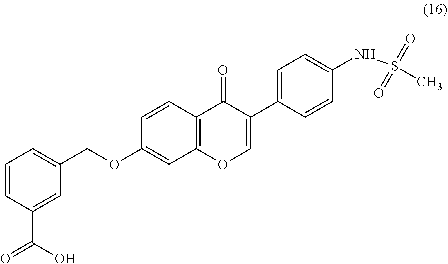 [SMILES: CS(=O)(=O)Nc1ccc(-c2coc3cc(OCc4cccc(C(=O)O)c4)ccc3c2=O)cc1]

or a pharmaceutically acceptable salt, ester, or a tautomer thereof.

Additionally, the present disclosure also provides a patient pack comprising at least one pharmaceutical composition as disclosed herein, and an information package or product insert containing directions on the method of using the pharmaceutical compositions.

Additional embodiments are described herein.

### BRIEF DESCRIPTION OF THE DRAWINGS

FIG. 1 depicts a schematic representation of the design of the prevention of acquisition of conditioned response study described in Example 4.

FIG. 2A, FIG. 2B, and FIG. 2C depict plots of results of the dose-effect study with compound (2) described in Example 4. FIG. 2A shows plots of the mean number of lever presses by the group of rats receiving vehicle (0), 9, 18, 36, and 72 mg compound (2) each day of the 10-day period of acquisition of the conditioned response; FIG. 2B depicts plots of the responses observed on days 1-5; FIG. 2C depicts plots of the responses observed on days 6-10.

### DETAILED DESCRIPTION

It is to be understood that the detailed descriptions provided herein, including the drawings, are exemplary and explanatory only and are not restrictive of this disclosure. The description is not limited to the specific compounds, compositions, methods, techniques, protocols, cell lines, assays, and reagents disclosed herein, as these may vary, but is also intended to encompass known variants of these specific embodiments.

It is also to be understood that the terminology used herein is intended to describe particular embodiments, and is in not intended to limit the scope as set forth in the appended claims. For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Further, it is understood that where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

### Abbreviations, Definitions and General Parameters

As used in the present specification, the following words and phrases are generally intended to have the meanings as set forth below, except to the extent that the context in which they are used indicates otherwise.

The term “ALDH-2 inhibitor” as used herein includes any compound that selectively inhibits the enzyme aldehyde dehydrogenase 2. Exemplary ALDH-2 inhibitor compounds include the isoflavone compound, daidzein (see e.g., U.S. Pat. Nos. 5,624,910, and 6,121,010), and its structurally related isoflavone derivative compounds (see e.g., U.S. Pat. Nos. 7,951,813, 8,158,810, and 8,673,966; Int'l Pat. Publ. Nos. WO2008/014497, WO2008/124532, WO2009/061924, WO2009/094028, and WO2013/033377), and compounds of Formula (I), which are structurally unrelated to the isoflavones, such as 2,6-dichloro-N-[4-(2-oxo-1,2-dihydro-pyridin-4-yl)-benzyl]-benzamide (see e.g., U.S. Pat. Nos. 8,558,001, 8,575,353, 9,000,015, 9,610,299; Int'l Pat. Publ. WO2013/006400).

The term “addiction” as used herein includes any substance use disorder including, but not limited to, substance misuse, substance dependence, substance addiction, and/or conditioned response behavior in a mammal resulting from a dopamine producing agent.

The term “dopamine producing agents” as used herein includes compounds capable of inducing a surge in dopamine levels in a mammal, including, but not limited to, opioids, amphetamines, nicotine, alcohol, other drugs of addiction, and foods (e.g., sugary foods).

The term “opioid” as used herein refers to any substance that activates an opioid receptor to produce a morphine-like effect.

The term “opioid medication” refers to a pharmaceutical compound or pharmaceutical composition that contains an opioid, including but not limited to alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

The term “therapeutically effective amount” refers to an amount that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “unit dosage form” refers to physically discrete units suitable as unitary dosages for human subjects and other mammals, each unit containing a predetermined quantity of active ingredient that produces the desired therapeutic effect, in association with a suitable pharmaceutical excipient (e.g., a tablet, capsule, or ampoule).

The term “active ingredient” refers to a compound in a pharmaceutical composition that has a pharmacological effect when administered to an organism (e.g., a mammal) and is intended to encompass not only the compound but also the pharmaceutically acceptable salts, pharmaceutically acceptable esters, hydrates, polymorphs, and prodrugs of such compound.

The term “prodrug” refers to a compound that includes a chemical group which, in vivo, can be converted and/or split off from the remainder of the molecule to provide for the active drug, a pharmaceutically acceptable salt thereof, or a biologically active metabolite thereof.

The term “combination dosage form” refers to a unit dosage form (e.g., single pill, tablet, capsule, ampoule, suppository, or other unit dosage form) that contains a combination of two or more active ingredients (e.g., ALDH-2 inhibitor and opioid medication).

The term “treatment” or “treating” means any administration of a compound of the disclosure to a mammal having a disease or susceptible to a disease for purposes including:
(i) preventing the disease, that is, causing the clinical symptoms of the disease not to develop;
(ii) inhibiting the disease, that is, arresting the development of clinical symptoms; and/or
(iii) relieving the disease, i.e. causing the regression of clinical symptoms.

The term “in combination with” as used in the context of administering the two or more active ingredients in a method of treatment (e.g., the dopamine-producing agent and the ALDH-2 inhibitor compound) includes administering the active ingredients separately (e.g., sequentially) or together (e.g., simultaneously).

The term “alkyl” refers to a monoradical branched or unbranched saturated hydrocarbon chain having from 1 to 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
(i) an alkyl group as defined above, having 1, 2, 3, 4 or 5 substituents, (typically 1, 2, or 3 substituents) selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkyl group as defined above that is interrupted by 1-10 atoms (e.g. 1, 2, 3, 4, or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
(iii) an alkyl group as defined above that has both 1, 2, 3, 4 or 5 substituents as defined above and is also interrupted by 1-10 atoms (e.g. 1, 2, 3, 4, or 5 atoms) as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (typically 1, 2, or 3 substituents), as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, typically having from 1 to 20 carbon atoms (e.g. 1-10 carbon atoms, or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, typically having 1, 2, 3, 4, 5, or 6 carbon atoms.

The term “substituted alkylene” refers to:
(i) an alkylene group as defined above having 1, 2, 3, 4, or 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkylene group as defined above that is interrupted by 1-10 groups (e.g. 1, 2, 3, 4, or 5 groups) independently chosen from —O—, —S—, sulfonyl, —C(O)—, —C(O)O—, —C(O)N—, and —NR^{a}, where R^{a} is chosen from hydrogen, optionally substituted alkyl, cycloalkyl, cycloalkenyl, aryl, heteroaryl and heterocyclyl; or
(iii) an alkylene group as defined above that has both 1, 2, 3, 4 or 5 substituents as defined above and is also interrupted by 1-10 groups as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers(—CH₂CH(CO₂H)CH₂—), ethoxyethyl (—CH₂CH₂O—CH₂CH₂—), ethylmethylaminoethyl (—CH₂CH₂—N(CH₃)—CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkoxy” refers to the group R—O—, where R is optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are alkyl-O— and include, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl as defined above. This term is exemplified by groups such as methoxy, ethoxy, n-propoxy, iso-propoxy, n-butoxy, iso-butoxy, t-butoxy, n-hexyloxy, and the like.

The term “alkylthio” refers to the group R—S—, where R is as defined for alkoxy.

The term “alkenyl” refers to a monoradical of a branched or unbranched unsaturated hydrocarbon group typically having from 2 to 20 carbon atoms (more typically from 2 to 10 carbon atoms, e.g. 2 to 6 carbon atoms) and having from 1 to 6 carbon-carbon double bonds, e.g. 1, 2, or 3 carbon-carbon double bonds. Typical alkenyl groups include ethenyl (or vinyl, i.e. —CH═CH₂), 1-propylene (or allyl, —CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, typically having from 2 to 20 carbon atoms (more typically from 2 to 10 carbon atoms, e.g. 2 to 6 carbon atoms) and having from 1 to 6 carbon-carbon triple bonds e.g. 1, 2, or 3 carbon-carbon triple bonds. Typical alkynyl groups include ethynyl (—C≡CH), propargyl (or propynyl, —C≡CCH₃), and the like. In the event alkynyl is attached to nitrogen, the triple bond cannot be alpha to the nitrogen.

The term “substituted alkynyl” refers to an alkynyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, cycloalkyl, aryl, heteroaryl, heterocyclyl or where both R groups are joined to form a heterocyclic group (e.g., morpholino). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “ester” or “carboxyester” refers to the group —C(O)OR, where R is alkyl, cycloalkyl, aryl, heteroaryl, or heterocyclyl, which may be optionally further substituted by alkyl, alkoxy, halogen, CF₃, amino, substituted amino, cyano, or —S(O)R^{a}, in which Ra is alkyl, aryl, or heteroaryl and n is 0, 1 or 2.

The term “acylamino” refers to the group —NRC(O)R where each R is independently hydrogen, alkyl, aryl, heteroaryl, or heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2.

The term “acyloxy” refers to the groups —OC(O)-alkyl, —OC(O)-cycloalkyl, —OC(O)-aryl, —OC(O)-heteroaryl, and —OC(O)-heterocyclyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl, fluorenyl, and anthryl). Typical aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl, —C(O)O-cycloalkyl, where alkyl and cycloalkyl are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and bicyclo[2.2.1]heptane, or cyclic alkyl groups to which is fused an aryl group, for example indan, and the like.

The term “cycloalkenyl” refers to cyclic alkyl groups of from 3 to 20 carbon atoms having a single cyclic ring or multiple condensed rings and having at least one double bond and preferably from 1 to 2 double bonds.

The terms “substituted cycloalkyl” and “susbstituted cycloalkenyl” refer to cycloalkyl or cycloalkenyl groups having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂— heteroaryl. The term “substituted cycloalkyl” also includes cycloalkyl groups wherein one or more of the annular carbon atoms of the cycloalkyl group is a carbonyl group (i.e. an oxygen atom is oxo to the ring). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “alkoxycarbonylamino” refers to a group —NHC(O)OR in which R is optionally substituted alkyl.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which R each R is independently an optionally substituted alkyl.

The term “azido” refers to a group

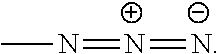

The term “hydroxyl” or “hydroxyl” refers to a group —OH.

The term “arylthio” refers to the group —S-aryl.

The term “heterocyclylthio” refers to the group —S-heterocyclyl.

The term “alkylthio” refers to the group —S-alkyl.

The term “aminosulfonyl” refers to the group —SO₂NRR, wherein each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl.

The term “aminocarbonylamino” refers to the group —NR^{c}C(O)NRR, wherein R^{c} is hydrogen or alkyl and each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl.

The term “heterocyclooxy” refers to the group —O-heterocyclyl.

The term “alkoxyamino” refers to the group —NHOR in which R is optionally substituted alkyl.

The term “hydroxyamino” refers to the group —NHOH.

The term “heteroaryl” refers to a group comprising single or multiple rings comprising 1 to 15 carbon atoms and 1 to 4 heteroatoms selected from oxygen, nitrogen, and sulfur within at least one ring. The term “heteroaryl” is generic to the terms “aromatic heteroaryl” and “partially saturated heteroaryl.”The term “aromatic heteroaryl” refers to a heteroaryl in which at least one ring is aromatic. Examples of aromatic heteroaryls include pyrrole, thiophene, pyridine, quinoline, pteridine. The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, and the like.

Unless otherwise constrained by the definition for the heteroaryl substituent, such heteroaryl groups can be optionally substituted with 1 to 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl (an alkyl ester), arylthio, heteroaryl, heteroarylthio, heterocyclylthio, thiol, alkylthio, aryl, aryloxy, aralkyl, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Such heteroaryl groups can have a single ring (e.g., pyridyl or furyl) or multiple condensed rings (e.g., indolizinyl, benzothiazole, or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “heterocyclyl,” “heterocycle,” or “heterocyclic” refers to a monoradical saturated group having a single ring or multiple condensed rings, having from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl,—SO-heteroaryl, —SO-alkyl, SO-aryl and —SO2-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Preferred heterocyclics include tetrahydrofuranyl, morpholino, piperidinyl, and the like.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” or “oxo” refers to a group —C(O)—.

The term “thiocarbonyl” refers to a group —C(S)—.

The term “carboxy” refers to a group —C(O)—OH.

The term “optional” or “optionally” mean that the subsequently described event or circumstance may or may not occur, and that the description includes instances where said event or circumstance occurs and instances in which it does not.

The term “substituted” includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to substituted aryl-(substituted aryl)-substituted aryl.

A compound of a given formula (e.g. the “compound of Formula (I)”) is intended to encompass the compounds of the disclosure, and the pharmaceutically acceptable salts, pharmaceutically acceptable esters, hydrates, polymorphs, and prodrugs of such compounds.

Additionally, the compounds of the disclosure may possess one or more asymmetric centers, and can be produced as a racemic mixture or as individual enantiomers or diastereoisomers. The number of stereoisomers present in any given compound of a given Formula depends upon the number of asymmetric centers present (there are 2n stereoisomers possible where n is the number of asymmetric centers). The individual stereoisomers may be obtained by resolving a racemic or non-racemic mixture of an intermediate at some appropriate stage of the synthesis, or by resolution of the compound by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

The term “isomers” means different compounds that have the same molecular formula. Isomers include stereoisomers, enantiomers, and diastereomers.

The term “stereoisomers” means isomers that differ only in the way the atoms are arranged in space.

The term “enantiomers” means a pair of stereoisomers that are non-superimposable mirror images of each other. A 1:1 mixture of a pair of enantiomers is a “racemic” mixture. The term “(±)” is used to designate a racemic mixture where appropriate.

The term “diastereoisomers” means stereoisomers that have at least two asymmetric atoms, but which are not mirror-images of each other.

Absolute stereochemistry is specified herein according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or levorotary) that they rotate the plane of polarized light at the wavelength of the sodium D line.

Some of the compounds of the present disclosure exist as “tautomeric isomers” or “tautomers.” “Tautomeric isomers” or “tautomers” are isomers that are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Non-limiting examples of amide-comprising and imidic acid-comprising tautomers are shown below:

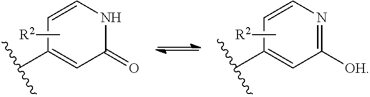

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by combining a compound and a solvent.

The term “hydrate” refers to the complex formed by combining a compound and water.

The term “pharmaceutically acceptable salt” of a given compound refers to salts that retain the biological effectiveness and properties of the given compound, and which are not biologically or otherwise undesirable. In many cases, the compounds of this disclosure are capable of forming pharmaceutically acceptable acid and/or base salts by virtue of the presence of amino and/or carboxyl groups or groups similar thereto.

Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts also may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “pharmaceutically acceptable carrier” or “pharmaceutically acceptable excipient” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Any formula or structure given herein, including Formula (I) compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, ¹²⁵I. Various isotopically labeled compounds of the present invention, for example those into which radioactive isotopes such as ³H, ¹³C, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients.

Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula (I).

The concentration of such a heavier isotope, specifically deuterium, may be defined by an isotopic enrichment factor. In the compounds of this invention any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this invention any atom specifically designated as a deuterium (D) is meant to represent deuterium.

In the description, including the examples, all temperatures are in degrees Celsius (° C.), unless otherwise stated, and abbreviations and acronyms have the following meanings:

| Abbreviation | Meaning |
|---|---|
| ° C. | Degree Celsius |
| 5-HIAA | 5-Hydroxyindoleacetic acid |
| 5-HIAL | 5-Hydroxyindoleacetaldehyde |
| 5-HT | 5-Hydroxytryptamine (serotonin) |
| 5-HTOL | 5-Hydroxytryptophol |
| Ae | Enzyme activities measured in the presence of a test |
| | compound |
| AIDS | Acquired immune deficiency syndrome |
| ALDH-2 | Human mitochondrial aldehyde dehydrogenase |
| Ao | Enzyme activities measured in the absence of a test |
| | compound |
| BHA | Butylated hydroxy anisole |
| BOC | tert-Butoxycarbonyl |
| BOP | Benzotriazolyl-N-hydroxytris(dimethyamino)phosphonium |
| | hexafluorophosphate |
| Cbz | Benzyl carbamate |
| cm | centimeter |
| d | Doublet |
| dd | Doublet of doublets |
| DA | Dopamine |
| DCC | Dicyclohexyl carbodiimide |
| DCM | Dichloromethone |
| DIC | Diisopropyl carbodiimide |
| DIEA | N,N-Diisopropylethylamine |
| DMF | Dimethylformamide |
| DMSO | Dimethylsulfoxide |
| dt | Doublet of triplets |
| EDTA | Ethylenediaminetetraacetic acid |
| equiv/eq | Equivalents |
| EtOAc | Ethyl acetate |
| EtOH | Ethanol |
| FR | Fixed ratio |
| g | Grams |
| HATU | O-(7-Azabenzotriazol-1-yl)-N,N,N′,N′-tetramethyluronium |
| | hexafluorophosphate |
| HBTU | O-Benzotriazole-N,N,N′,N′-tetramethyl-uronium- |
| | hexafluoro-phosphate |
| HPLC | High-performance liquid chromatography |
| hrs/h | Hours |
| Hz | Hertz |
| IC50 | The half maximal inhibitory concentration |
| IIDQ | 1-Isobutoxycarbonyl-2-isobutoxy-1,2-dihydro quinone |
| ip | Intraperitoneal |
| iv | Intravenous |
| J | Coupling constant |
| Kg | Kilogram |
| L | Liter |
| LAD | Low alcohol-drinking rat |
| LCMS/LC-MS | Liquid chromatography-mass spectrometry |
| LG | Leaving group |
| M | Molar |
| m/z | mass-to-charge ratio |
| M+ | Mass peak |
| M + H | Mass peak plus hydrogen |
| M + Na | Mass peak plus sodium |
| MAO | Monoamine oxidase |
| Me | Methyl |
| mg | Milligram |
| MHz | Megahertz |
| min | Minute |
| ml/mL | Milliliter |
| mM | Millimolar |
| mmol | Millimole |
| MOM | Methoxylmethyl |
| MS | Mass spectroscopy |
| NAD | Nicotinamide Adenine Dinucleotide |
| NaPPi | Sodium pyrophosphate |
| NIH | National Institute of Health |
| NMM | N-Methylmorpholine |
| NMR | Nuclear magnetic resonance |
| NP | Alcohol non-preferring rat |
| OCD | Obsessive compulsive disorder |
| PG | Protecting group |
| Ph | Phenyl |
| PyBOP | (Benzotriazol-1-yloxy)tripyrrolidinophosphonium |
| | hexafluorophosphate |
| q.s. | Quantity sufficient to achieve a stated function |
| RT/rt/R.T | Room temperature |
| s | Second |
| s | Singlet |
| SA | Self-administration |
| sc | Subcutaneous |
| SEM | Standard error of means |
| t | Triplet |
| TEA | Triethylamine |
| TES | Triethylsilyl |
| TFA | Trifluoroacetic acid |
| THF | Tetrahydrofuran |
| TIPS | Triisopropylsilyl |
| TKK | TKK buffer |
| TLC | Thin layer chromatography |
| TMS | Trimethylsilyl |
| TO | Time out |
| Tris | tris(hydroxymethyl)aminomethone |
| δ | Chemical shift |
| μg | Microgram |
| μL/μl | Microliter |
| μM | Micromolar |
| μmol | Micromole |

### Dopamine-Producing Agents

Many substances, including medications, comprise dopamine producing agents as active ingredients. It is now well-established that such substances when administered to mammals (e.g., humans) induce surges in dopamine levels (either directly or indirectly) that can result in the acquisition of a conditioned response that leads to the deleterious side-effect of addiction (e.g., misuse, dependence, abuse). The methods of treatment provided in the present disclosure are useful in reducing or preventing the acquisition of a conditioned response, and addiction, that can result from the use of any substance that contains a dopamine-producing agent, or is otherwise capable of inducing a surge in dopamine levels in the subject to which it is administered. Well-known dopamine producing agents include opioids, amphetamines, nicotine, alcohol, other drugs of addiction, and foods (e.g., sugary foods).

Opioid medications constitute a class of dopamine producing agents that are widely used to treat humans, for example as analgesics for the treatment of post-surgical pain and/or chronic pain. Yet treatment with opioid medications results in a high risk of patient addiction due to the dopamine surges caused by the compounds. Accordingly, the present disclosure contemplates that the methods of treatments disclosed herein can be used with any method of treatment that includes administration of an opioid medication to a mammal, and particularly a human. Similarly, it is contemplated that the pharmaceutical compositions of the present disclosure that include dopamine-producing agent can include an opioid medication.

A wide-range of opioid medications are well-known in the art. It is contemplated that any of these known opioid medications can be used in the methods and pharmaceutical compositions of the present disclosure. In particular, an opioid medication useful in the methods and compositions can be selected from any of the following: alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

### ALDH-2 Inhibitor Compounds

As described elsewhere herein, ALDH-2 inhibitor compounds have been shown to reduce or prevent dopamine surges in mammals resulting from the intake of dopamine-producing agents such cocaine, nicotine, and alcohol, and thereby reduce the likelihood of addiction relapse. The methods and compositions of the present disclosure are useful for the reduction and/or prevention of acquisition of a conditioned response, such as addiction, in mammals to a substance, including a medication containing a dopamine-producing agent, through the administration of an ALDH-2 inhibitor in combination with the substance. ALDH-2 inhibitor compounds useful in the methods and compositions of the present disclosure can include any of the compounds well-known in the art as ALDH-2 inhibitors including, but not limited to, daidzein (compound (15)), or its pharmaceutically acceptable salts, esters, or a tautomer thereof.

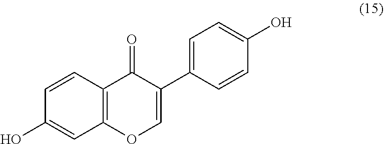 [SMILES: O=c1c(-c2ccc(O)cc2)coc2cc(O)ccc12]

ALDH-2 inhibitor compounds useful in the methods and compositions of the present disclosure can include the isoflavone compounds structurally related to daidzein, such as 3-{[3-(4-aminophenyl)-4-oxochromen-7-yloxy]methyl}benzoic acid (compound (16)), or its pharmaceutically acceptable salts, esters, or a tautomer thereof.

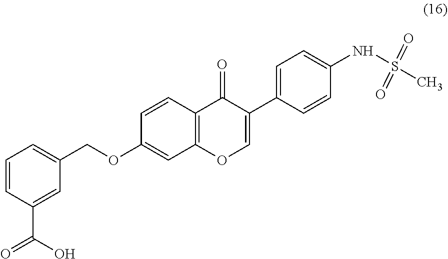 [SMILES: CS(=O)(=O)Nc1ccc(-c2coc3cc(OCc4cccc(C(=O)O)c4)ccc3c2=O)cc1]

Additional ALDH-2 inhibitor compounds comprising an isoflavone structure that are useful in the methods and compositions of the present disclosure are described in U.S. Pat. Nos. 5,624,910, 6,121,010 7,951,813, 8,158,810, and 8,673,966, and Int'l Pat. Publ. Nos. WO2008/014497, WO2008/124532, WO2009/061924, WO2009/094028, and WO2013/033377, each of which is hereby incorporated by reference herein.

ALDH-2 inhibitor compounds useful in the methods and compositions of the present disclosure can include any of the ALDH-2 inhibitor compounds that are structurally unrelated to daidzein and the other isoflavones. These include the ALDH-2 inhibitor compounds described in U.S. Pat. Nos. 8,558,001, 8,575,353, 9,000,015, 9,610,299, Int'l Pat. Publ. WO2013/006400, each of which is hereby incorporated by reference herein. Accordingly, in some embodiments of the methods and compositions of the present disclosure, the ALDH-2 inhibitor compound used is a compound of Formula (I):

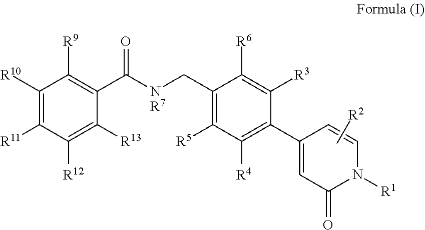

wherein:

R¹ is hydrogen, optionally substituted C₁₋₆ alkyl, —CH₂OH, —CH₂OP(O)(OR²⁰)(OR²¹);

R² is hydrogen, optionally substituted C₁₋₆ alkyl, cycloalkyl, or halo;

each of R³, R⁴, R⁵, R⁶, R⁹, R¹⁰, R¹¹, R¹² and R¹³ is independently hydrogen, hydroxyl, —OP(O)(OR²⁰)(O²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O-(C₁ to C₆-alkyl), cyano, halo, —SO₂NR²⁴R²⁵; or —NR²⁴R²⁵;

R⁷ is hydrogen or optionally substituted C₁₋₆ alkyl;

each of R²⁰ and R²¹ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and

each of R²⁴ and R²⁵ is independently chosen from hydrogen or C₁₋₆ alkyl or when combined together with the nitrogen to which they are attached form a heterocycle; or

a pharmaceutically acceptable salt, ester, single stereoisomer, mixture of stereoisomers, or a tautomer thereof.

The naming and numbering of the compounds of Formula (I) is illustrated with a representative compound (1):

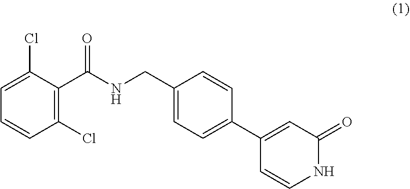 [SMILES: O=C(NCc1ccc(-c2cc[nH]c(=O)c2)cc1)c1c(Cl)cccc1Cl]

namely: 2,6-dichloro-N-[4-(2-oxo-1,2-dihydro-pyridin-4-yl)-benzyl]-benzamide. In certain embodiments, R¹ is hydrogen. In certain embodiments, R¹ is C₁₋₆ alkyl. In certain embodiments, R¹ is methyl. In certain embodiments, R¹ is —CH₂OP(O)(OR²⁰)(OR²¹); and each of R²⁰ and R²¹ is independently Na⁺, Li⁺, K⁺, or hydrogen. In certain embodiments, at least one of R¹, R⁹, R¹⁰, R¹¹, R¹², R¹³ is not hydrogen. In other embodiments, at least two of R¹, R⁹, R¹⁰, R¹¹, R¹², R¹³ is not hydrogen.

In certain embodiments, R² is hydrogen. In certain embodiments, R² is C₁₋₆ alkyl. In certain embodiments, R² is methyl. In certain embodiments, R² is selected from the group consisting of ethyl, n-propyl, iso-propyl, n-butyl, iso-butyl, t-butyl, and n-hexyl. In certain embodiments, R² is halo. In certain embodiments, R² is fluoro. In certain embodiments, R² is chloro. In certain embodiments, R² is bromo. In certain embodiments, R² is iodo.

In certain embodiments, each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted C₁₋₆ alkyl, optionally substituted C₃₋₈ cycloalkyl, optionally substituted C₁₋₆ alkoxy, —O—(C₁ to C₆-alkyl)-O-(C₁ to C₆-alkyl), —C(O)NH₂, cyano, or halo. In certain embodiments, each of R³, R⁴, R⁵, and R⁶ is independently hydrogen, C₁₋₆ alkyl, or halo. In certain embodiments, one of R³, R⁴, R⁵, or R⁶ is C₁₋₆ alkyl or halo. In certain embodiments, one of R³, R⁴, R⁵, or R⁶ is selected from the group consisting of ethyl, n-propyl, iso-propyl, n-butyl, iso-butyl, t-butyl, and n-hexyl. In certain embodiments, one of R³, R⁴, R⁵, or R⁶ is methyl. In certain embodiments, one of R³, R⁴, R⁵, or R⁶ is fluoro. In certain embodiments, one of R³, R⁴, R⁵, or R⁶ is chloro. In certain embodiments, one of R³, R⁴, R⁵, or R⁶ is fluoro. In certain embodiments, one of R³, R⁴, R⁵, or R⁶ is iodo.

In certain embodiments, R⁷ is hydrogen. In certain embodiments, R⁷ is C₁₋₆ alkyl. In certain embodiments, R⁷ is selected from the group consisting of ethyl, n-propyl, iso-propyl, n-butyl, iso-butyl, t-butyl, and n-hexyl. In certain embodiments, R⁷ is methyl.

In certain embodiments, at least one of R⁹ and R¹³ is not hydrogen. In certain embodiments, at least one of R⁹ and R¹³ is halo or C₁₋₆ alkyl. In certain embodiments, at least one of R⁹ and R¹³ is selected from the group consisting of ethyl, n-propyl, iso-propyl, n-butyl, iso-butyl, t-butyl, and n-hexyl. In certain embodiments, at least one of R⁹ and R¹³ is independently chloro, fluoro, or methyl. In certain embodiments, at least one of R⁹ and R¹³ is bromo. In certain embodiments, at least one of R⁹ and R¹³ is iodo. In certain embodiments, R⁹ and R¹³ are independently halo or C₁₋₆ alkyl. In certain embodiments, R⁹ and R¹³ are independently chloro, fluoro, or methyl. In certain embodiments, R⁹ and R¹³ are chloro. In certain embodiments, R⁹ and R¹³ are methyl.

In certain embodiments, each of R¹⁰ and R¹² is independently hydrogen, halo, or C₁₋₆ alkyl. In certain embodiments, each of R¹⁰ and R¹² is independently ethyl, n-propyl, iso-propyl, n-butyl, iso-butyl, t-butyl, and n-hexyl. In certain embodiments, each of R¹⁰ and R¹² is independently hydrogen, chloro, fluoro, or methyl. In certain embodiments, each of R¹⁰ and R¹² is independently bromo. In certain embodiments, each of R¹⁰ and R¹² is independently iodo. In certain embodiments, each of R¹⁰ and R¹² is independently fluoro. In certain embodiments, each of R¹⁰ and R¹² is independently chloro. In certain embodiments, R¹⁰ and R¹² are hydrogen.

In certain embodiments, R¹¹ is hydrogen. In certain embodiments, R¹¹ is —O-(C₁ to C₆-alkyl)-O-(C₁ to C₆-alkyl). In certain embodiments, R¹¹ is —OCH₂CH₂OCH₃. In certain embodiments, R¹¹ is independently ethyl, n-propyl, iso-propyl, n-butyl, iso-butyl, t-butyl, and n-hexyl. In certain embodiments, R¹¹ is halo. In certain embodiments, R¹¹ is fluoro. In certain embodiments, R¹¹ is chloro. In certain embodiments, R¹¹ is bromo. In certain embodiments, R¹¹ is iodo.

In certain embodiments,

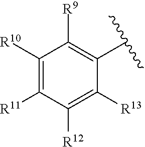

is selected from the group consisting of:

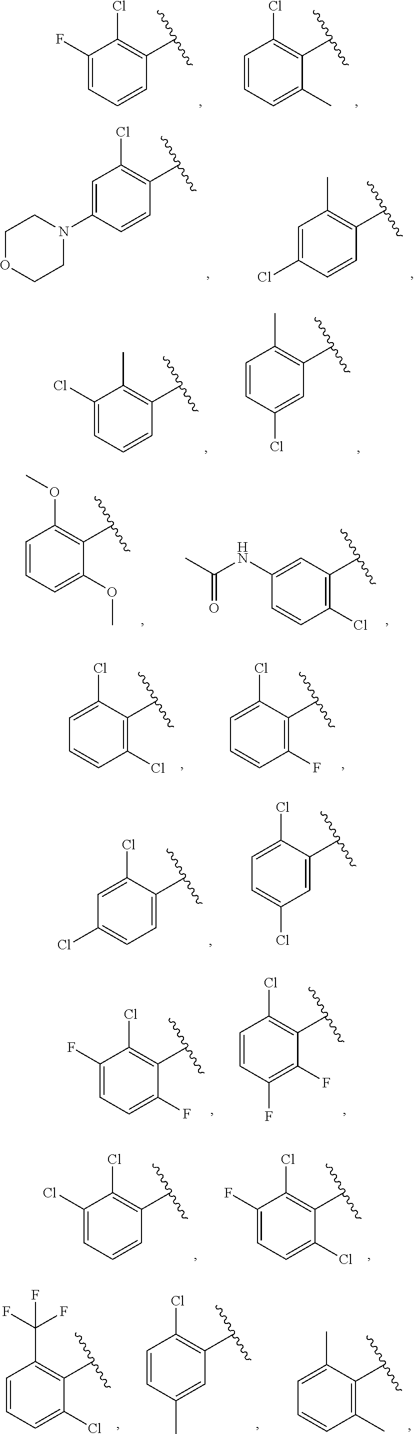

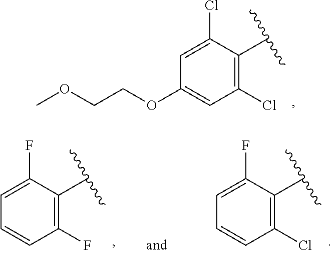

In certain embodiments, R¹ is hydrogen, methyl, or —CH₂OP(O)(OR²⁰)(OR²¹); R² is hydrogen, methyl, or fluoro; each of R³ and R⁴ is independently hydrogen or methyl; each of R⁵ and R⁶ is independently hydrogen or fluoro; R⁷ is hydrogen; R⁹ is hydrogen, chloro, fluoro, or methyl; R¹⁰ is hydrogen or fluoro; R¹¹ is hydrogen or —OCH₂CH₂OCH₃; R¹² is hydrogen or fluoro; R¹³ is hydrogen, chloro, fluoro, or methyl; and each of R²⁰ and R²¹ is independently Na⁺, Li⁺, K⁺, or hydrogen.

In certain embodiments, the ALDH-2 inhibitor compound of Formula (I) is selected from the group consisting of the compounds (1)-(14) listed in Table 1. As described in U.S. Pat. No. 8,558,001, each of these compounds exhibits high, selective inhibition of the human ALDH-2 enzyme, with IC₅₀ values of less than 1 μm, and relatively low inhibitory activity toward the MAO-A and MAO-B pathway enzymes, with IC₅₀ values of >130 μm. It should be noted that high IC₅₀ value for compound (2) is due to it being a phosphoric acid adduct prodrug of compound (1). Thus, compound (2) undergoes in vivo cleavage of the phosphoric acid group to yield compound (1).

TABLE 1
Exemplary ALDH-2 Inhibitor Compounds of Formula (I)

| | | IC50 | IC50 | IC50 |
|---|---|---|---|---|
| Compound | | ALDH-2 | hMAO-A | hMAO-B |
| No. | Compound Name | (nm) | (μm) | (μm) |
| (1) | 2,6-dichloro-N-[4-(2-oxo-1,2-dihydro-pyridin- | 102 | >130 | >130 |
| | 4-yl)-benzyl]-benzamide | | | |
| (2) | phosphoric acid mono-(4-{4-[(2,6- | >10000.00 | >129.51 | >130 |
| | dichloro-benzoylamino)-methyl]-phenyl}-2- | | | |
| | oxo-2H-pyridin-1-ylmethyl) ester | | | |
| (3) | 2,6-dichloro-4-(2-methoxyethoxy)-N-(4-(2- | 63 | >130 | >130 |
| | oxo-1,2-dihydropyridin-4- | | | |
| | yl)benzyl)benzamide | | | |
| (4) | 2-chloro-3-fluoro-N-(4-(2-oxo-1,2- | 215 | >130 | >130 |
| | dihydropyridin-4-yl)benzyl)benzamide | | | |
| (5) | 2-chloro-6-methyl-N-(4-(2-oxo-1,2- | 23 | >130 | >130 |
| | dihydropyridin-4-yl)benzyl)benzamide | | | |
| (6) | 2,6-dimethyl-N-(4-(2-oxo-1,2-dihydropyridin- | 166 | >130 | >130 |
| | 4-yl)benzyl)benzamide | | | |
| (7) | 2,6-dichloro-N-[4-(6-methyl-2-oxo-1,2- | 1113 | >130 | >130 |
| | dihydro-pyridin-4-yl)-benzyl]-benzamide | | | |
| (8) | 2-chloro-3,6-difluoro-N-(4-(2-oxo-1,2- | 464 | >130 | >130 |
| | dihydropyridin-4-yl)benzyl)benzamide | | | |
| (9) | 2,6-dichloro-N-(3-methyl-4-(2-oxo-1,2- | 480 | >130 | >130 |
| | dihydropyridin-4-yl)benzyl)benzamide | | | |
| (10) | 2,6-dichloro-N-(4-(1-methyl-2-oxo-1,2- | 2093 | >130 | >130 |
| | dihydropyridin-4-yl)benzyl)benzamide | | | |
| (11) | 2,6-difluoro-N-(4-(2-oxo-1,2-dihydropyridin- | 890 | >130 | >130 |
| | 4-yl)benzyl)benzamide | | | |
| (12) | 2-chloro-6-fluoro-N-(4-(2-oxo-1,2- | 379 | >130 | >130 |
| | dihydropyridin-4-yl)benzyl)benzamide | | | |
| (13) | 2,6-dichloro-N-(2-fluoro-4-(2-oxo-1,2- | 304 | >130 | >130 |
| | dihydropyridin-4-yl)benzyl)benzamide | | | |
| (14) | 2,6-dichloro-N-(4-(5-fluoro-2-oxo-1,2- | 25 | >130 | >130 |
| | dihydropyridin-4-yl)benzyl)benzamide | | | |

In certain embodiments, the compound of Formula (I) is compound (1):

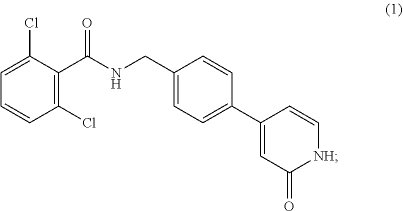 [SMILES: O=C(NCc1ccc(-c2cc[nH]c(=O)c2)cc1)c1c(Cl)cccc1Cl]

or a pharmaceutically acceptable salt, ester, single stereoisomer, mixture of stereoisomers, or tautomer thereof.

In certain embodiments, the compound of Formula (I) is compound (2):

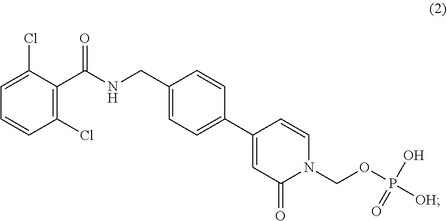 [SMILES: O=C(NCc1ccc(-c2ccn(COP(=O)(O)O)c(=O)c2)cc1)c1c(Cl)cccc1Cl]

or a pharmaceutically acceptable salt, ester, single stereoisomer, mixture of stereoisomers, or tautomer thereof. As noted above, compound (2) is an exemplary prodrug compound of Formula (I). It loses generates the free amide (pyridine) compound (1) in vivo as a metabolite. Accordingly, one of ordinary skill in the art can synthesize other prodrugs of compounds of Formula (I) based on the disclosure herein and synthetic methods well-known in the art.

### Preparation of the Compounds of Formula (I)

The compounds of Formula (I) can be prepared from readily available starting materials using methods and procedures known in the art. In particular, the disclosure of U.S. Pat. No. 8,558,001 (Cannizzaro et al.) issued Oct. 15, 2013, which is hereby incorporated by reference herein, provides general synthetic strategies for preparing compounds of Formula (I), and also exemplifies specific synthesis protocols that can be use to prepare the compounds (1), (2), (3), (4), (5), (6), (7), (8), (9), (10), (11), (12), (13), and (14) described herein and listed above in Table 1. Further, the synthetic protocol for the preparation of compounds (1) and (2) is provided below in the Examples of the present disclosure.

Briefly, the compounds of Formula (I) may be prepared according to the synthetic sequence shown in Scheme I

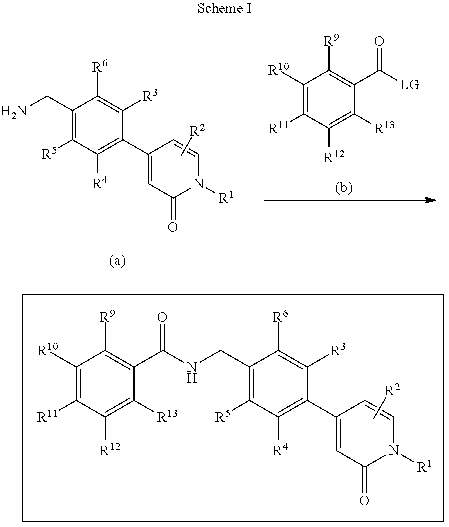

wherein, substituents R¹ through R²⁷, X¹, Y¹, Z¹ and Z² are as defined herein; LG is a leaving group (e.g., halo, hydroxyl, alkoxy, OSO₂ CF₃, N₂⁺, etc.); PG is a protecting group (e.g., t-butyl, t-butyl carbamate (BOC), etc.); and Z² is (OH)₂, (OMe)₂, F³⁻, or (OR^{H})(OR^{J}), wherein OR^{H} and OR^{J} may combine with boron to form a cyclic arylboronic ester moiety or cyclic alkylboronic ester moiety as described herein (e.g., 4,4,5,5-tetramethyl-1,3,2-dioxaboronic ester, catechol dioxaboronic ester, etc.); wherein R17 is an optionally substituted alkylene moiety of 1-6 carbon atoms.

The Scheme I reactants (a) and (b) are commercially available or can be prepared by means well known in the art. In general, the reactants (a) and at least one molar equivalent, and preferably a slight excess (e.g., 1.2 to 1.5 molar equivalents) of (b), as shown in Scheme I, are combined under standard reaction conditions in an inert solvent, such as dimethylformamide (DMF), at a temperature of about 25° C. until the reaction is complete, generally about 16 hours. Standard reaction conditions may comprise the use of a molar excess of suitable base, such as sodium or potassium hydroxide, triethylamine, diisopropylethylamine, N-methylmorpholine (NMM), or pyridine, or in some cases where LG is hydroxyl, a peptide coupling reagent, such as O-(7-azabenzotriazol-1-yl)-N,N,N′,N′-tetra methyluronium hexafluorophosphate (HATU), may be used. When the reaction is substantially complete, the product is subjected, if necessary, to a deprotection sequence under standard reaction conditions (e.g., THF, CH₂Cl₂, or the like, a molar excess of acid such as acetic acid, formic acid, trifluoroacetic acid, or the like as described herein) to yield isolated by conventional means. Further alternative synthetic methods for preparing compounds of Formula (I) are described in the synthetic sequences of Schemes II-V as disclosed in U.S. Pat. No. 8,558,001.

It will be appreciated that where typical or preferred process conditions (i.e., reaction temperatures, times, mole ratios of reactants, solvents, pressures, etc.) are disclosed in U.S. Pat. No. 8,558,001, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be necessary to prevent certain functional groups from undergoing undesired reactions. The term “protecting group” or “PG,” as used herein, is meant that a particular functional moiety, e.g., O, S, or N, is temporarily blocked so that a reaction can be carried out selectively at another reactive site in a multifunctional compound. “Protecting groups” or “PGs,” as used herein, are well known in the art and include those described in detail in Protective Groups in Organic Synthesis, Fourth Ed., Greene, T. W. and Wuts, P. G., Eds., John Wiley & Sons, New York: 2007, the entire contents of which are hereby incorporated by reference, and references cited therein.

The starting materials for the synthetic reactions Schemes I-V as disclosed in U.S. Pat. No. 8,558,001 are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Methods of Use

The present disclosure provides methods of use and treatment in which a substance that produces a conditioned response, such as a medication containing dopamine-producing agent (e.g., opioid medication), is administered in combination with an ALDH-2 inhibitor (e.g., compound of Formula (I)). Such methods act to reduce or prevent the acquisition of a conditioned response resulting in addiction to the substance, such as a medication, in the subject being treated with or otherwise using it. While not wishing to be bound by theory, ALDH-2 inhibitors (such as the compounds of Formula (I)) are known to be effective in reducing or preventing surges in dopamine levels caused by administration of a substances containing a dopamine-producing agent. It is believed that as a consequence of this ability of ALDH-2 inhibitors to reduce surges in dopamine, they also can reduce or prevent the acquisition of conditioned response or addiction in a subject, even a subject not previously exposed to the substance or medication containing the dopamine-producing agent. Based on this proposed mechanism of action, the ALDH-2 inhibitors (such as the compounds of Formula (I)) can be administered in combination with substances that produce a conditioned response, such as medications containing dopamine-producing agents, as in any of the methods of treatment provided herein, and thereby reduce or prevent addiction in a patient receiving the treatment.

Accordingly, the methods of the present disclosure comprise administering to a mammal in need thereof a therapeutically effective dose of an ALDH-2 inhibitor in combination with a substance that produces a conditioned response, such as a therapeutically effective dose of a medication that comprises a dopamine-producing agent. The two active ingredients (ALDH-2 inhibitor and substance) can be administered in combination with each other either separately or together (e.g., simultaneously). If administered separately, however, it is contemplated that the ALDH-2 inhibitor compound and substance, such as medication, be administered close enough in time such that levels of the ALDH-2 inhibitor present in the subject are sufficient to reduce or prevent the dopamine surge associated with the administration of the substance.

In some embodiments of the method, the administration in combination comprises administering the therapeutically effective dose of the ALDH-2 inhibitor prior to administration of the therapeutically effective dose of the medication comprising the dopamine-producing agent. In some embodiments, it is contemplated that the ALDH-2 is administered as a once-a-day dose. In some embodiments, the once-a-day dose is in a formulation (e.g., a tablet), that is self-administered by the subject or patient.

Additionally, medications comprising dopamine-producing agents, such as opioid medications, often require multiple doses administered to the subject throughout the day. Accordingly, it is contemplated in some embodiments of the methods, that the administration in combination comprises administering a therapeutically effective dose of the ALDH-2 inhibitor once-a-day, and administering a therapeutically effective dose of the medication comprising a dopamine-producing agent, such as an opioid medication, at least two or more times a day.

In some embodiment of the methods, the administration in combination comprises administering the therapeutically effective dose of the ALDH-2 inhibitor simultaneously with administration of the therapeutically effective dose of the substance, such as a medication comprising the dopamine-producing agent. For example, it is contemplated that a patient in thereof could self-administer an oral dosage form of the ALDH-2 inhibitor and an oral dosage form of the medication comprising a dopamine-producing agent simultaneously, e.g., two tablets taken at the same time.

In some embodiments of the methods, it is contemplated that the administration in combination comprises administering a pharmaceutical composition comprising both the therapeutically effective dose of the substance, such as medication containing the dopamine-producing agent, and the therapeutically effective dose of the ALDH-2 inhibitor compound, as well as a pharmaceutically acceptable carrier. In some embodiments, it is contemplated that this pharmaceutical composition comprising the two active ingredients of the medication and the ALDH-2 inhibitor is formulated in a unit dosage. Thus, in some embodiments of the method the administration in combination can comprise self-administration of a single unit dosage or combination dosage form, e.g., a single tablet, that comprises both active ingredients of the combination. Such embodiments include methods wherein the ALDH-2 inhibitor and the substance (e.g., medication comprising a dopamine-producing agent) are administered as combination dosage form.

It is contemplated that the method can be used with any disease-state that requires a course of treatment with a medication containing the dopamine-producing agent that is likely to increase the risk of addiction to the medication. For example, treatment of post-surgical or chronic pain with an opioid medication, where the patient typically self-administers the medication over a period of days, weeks, months, or longer. Thus, in some embodiments, the method is carried out wherein the mammal has undergone a surgical procedure and the medication is a post-surgical treatment. In some embodiments, the method is carried out wherein the mammal suffers from chronic pain and the medication is a treatment for pain.

As described above, in some embodiments of the method, the mammal (e.g., human patient) self-administers the pharmaceutically effective amount of the medication in combination with the pharmaceutically effective amount of the ALDH-2 inhibitor. Accordingly, in another aspect the present disclosure provides a patient pack comprising at least one pharmaceutical composition that comprises at least one of the active ingredients as described herein (e.g., a pharmaceutical composition comprising the medication and/or the ALDH-2 inhibitor) and an information package or product insert containing directions on the method of using the pharmaceutical compositions.

As noted above, the methods of the present disclosure can reduce or prevent acquisition of conditioned response, including addiction, to a medication comprising the dopamine-producing agent, even where the mammal has not previously been treated with or addicted to the medication. Accordingly, in some embodiments of the methods, prior to the administering of the ALDH-2 inhibitor in combination with the medication the mammal does not have an addiction to the dopamine-producing agent. In some embodiments, prior to the administering of the ALDH-2 inhibitor in combination with the medication, the mammal has not been treated with, used, or otherwise ingested the medication for at least 1 month, at least 3 months, at least 6 months, at least 1 year, or ever.

It is contemplated that the methods can be used to treat any mammal that is in need of a substance, such as a medication, comprising a dopamine-producing agent, and which therefore creates a risk of acquiring a conditioned response, such as addiction. In particular it is contemplated that the method can be used wherein the mammal is a human.

Accordingly, in some embodiments, the present disclosure provides a method of reducing or preventing in a mammal the acquisition of addiction to a substance, such as a medication, that comprises a dopamine-producing agent, wherein the method comprises administering to the mammal a therapeutically effective amount of an ALDH-2 inhibitor in combination with the substance, such as a medication; optionally, wherein the ALDH-2 inhibitor is a compound of Formula (I).

In some embodiments, the present disclosure provides a method of treating a mammal in need thereof with a medication that comprises a dopamine-producing agent, the method comprising administering to the mammal a therapeutically effective amount of the medication in combination with a therapeutically effective amount of an ALDH-2 inhibitor compound, optionally, wherein the ALDH-2 inhibitor is a compound of Formula (I).

In some embodiments, the present disclosure provides a method of treating pain in a mammal in need thereof, comprising administering to the mammal a therapeutically effective amount of an opioid medication in combination with a therapeutically effective amount of an ALDH-2 inhibitor compound.

In the various embodiments of the methods disclosed herein, the step of administering the medication containing the dopamine-producing agent in combination with the ALDH-2 inhibitor can comprise administering a pharmaceutical composition, wherein the pharmaceutical composition comprises the medication, the ALDH-2 inhibitor, and a pharmaceutically acceptable carrier.

Pharmaceutical Compositions

In some embodiments of the methods of the present disclosure, it is contemplated that the medication containing a dopamine-producing agent, and the ALDH-2 inhibitor compounds of Formula (I) are administered in combination with each other in the form of pharmaceutical compositions. When administered in separate doses, each dosage contains a therapeutically effective amount of the active ingredient (i.e., the medication, or the ALDH-2 inhibitor), or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants.

As noted above, in some embodiments of the methods of the present disclosure, the step of administering the medication containing the dopamine-producing agent in combination with the ALDH-2 inhibitor of Formula (I) can comprise administering a pharmaceutical composition, wherein the pharmaceutical composition is a combination composition that contains the medication (e.g., an opioid), the ALDH-2 inhibitor of Formula (I) (e.g., compound (2)), and a pharmaceutically acceptable carrier. Accordingly, in some embodiments the present disclosure also provides a pharmaceutical composition, wherein the composition comprises a therapeutically effective amount of a medication that comprises a dopamine-producing agent, a therapeutically effective amount of an ALDH-2 inhibitor, and a pharmaceutically acceptable carrier. In some embodiments, the combination pharmaceutical composition is in a unit dosage form, such as a combination dosage form that contains a combination of the active ingredients (e.g., ALDH-2 inhibitor and opioid medication) in a single dosage form.

Such pharmaceutical compositions can be prepared using methods well known in the pharmaceutical art (see, e.g., Remington's Pharmaceutical Sciences, Mace Publishing Co., Philadelphia, Pa. 17th Ed. (1985) and Modern Pharmaceutics, Marcel Dekker, Inc. 3rd Ed. (G.S. Banker & C.T. Rhodes, Eds.). Methods of preparing pharmaceutical compositions of ALDH-2 inhibitor compounds, such as compounds of Formula (I), are described in e.g., U.S. Pat. Nos. 7,951,813, 8,158,810, 8,673,966, 8,558,001, 8,575,353, 9,000,015, and 9,610,299, each of which is hereby incorporated by reference herein. Methods of preparing pharmaceutical compositions comprising medications containing dopamine-producing agents, and particularly, opioid medications are well-known in the art.

### Administering the Pharmaceutical Compositions

In the methods of the present disclosure it is contemplated that the pharmaceutical composition(s) comprising the medication containing a dopamine-producing agent and the ALDH-2 inhibitor, such as a compound of Formula (I), can be administered in combination with each other, either as single or multiple doses, and by any of the accepted modes of administration of active ingredients having similar utility. For example, as described in U.S. Pat. No. 8,558,001, a pharmaceutical composition comprising an ALDH-2 inhibitor compound of Formula (I) can be administered using a variety of different modes including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. A similarly wide range of modes are available for administering medications containing dopamine-producing agents, such as opioid medications.

One exemplary mode for administering useful in the methods of the present disclosure is parenteral, particularly by injection. The forms in which the novel compositions may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the active ingredients (e.g., compound of Formula (I)) in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the known methods of preparation include vacuum-drying and freeze-drying techniques which yield a powder of the active ingredient plus any additional desired ingredient from a previously sterile-filtered solution thereof.

Another exemplary route for administering that is useful in the methods of the present disclosure is oral. Oral administration may be via capsule, enteric coated tablets, or the like. Typically, in making the pharmaceutical compositions that include a medication containing a dopamine-producing agent and/or an ALDH-2 inhibitor, such as compound of Formula (I), the active ingredient(s) is diluted by an excipient and/or enclosed within a carrier in the form of a capsule, sachet, paper or other container. When the excipient serves as a diluent, it can be a solid, semi-solid, or liquid material (as above), which acts as a vehicle, carrier or medium for the active ingredient. Thus, the pharmaceutical composition(s) suitable for administering in the methods of the disclosure can be in the dosage form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Exemplary suitable excipients for the compositions of the present disclosure are well known in the art and include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical compositions useful in the methods of the present disclosure can be formulated so as to provide quick, sustained or delayed release of the relevant active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in e.g., U.S. Pat. Nos. 3,845,770; 4,326,525; 4,902514; and 5,616,345.

The pharmaceutical compositions useful in the methods of the present disclosure can also be formulated for administration via transdermal delivery devices (e.g., “patches”). Such transdermal patches may be used to provide continuous or discontinuous infusion of the pharmaceutical compositions in controlled amounts. The construction and use of transdermal patches for the delivery of pharmaceutical compositions, including opioid medications, is well known in the art. See, e.g., U.S. Pat. Nos. 5,023,252, 4,992,445 and 5,001,139. Such patches may be constructed for continuous, pulsatile, or on demand delivery of the pharmaceutical composition(s).

In some embodiments, the pharmaceutical composition(s) useful in the methods of the present disclosure are formulated in a unit dosage form.

The ALDH-2 inhibitor compounds useful in the methods of the present disclosure, e.g., compound of Formula (I) such as compound (2), are effective over a wide dosage range and is generally administered as a pharmaceutical composition in a pharmaceutically effective amount. In some embodiments, for oral administration, each dosage unit contains from about 10 mg to 1 g of a ALDH-2 inhibitor compound, such as compound of Formula (I), in some embodiments from 10 mg to 700 mg. In some embodiments, for parenteral administration, from 10 to 700 mg of an ALDH-2 inhibitor compound, such as compound of Formula (I), or in some embodiments, from about 50 mg to 300 mg.

Generally, in the methods of the disclosure, the amount of the ALDH-2 inhibitor compound, such as compound of Formula (I), to be administered in combination with the substance, such as a medication, containing a dopamine-producing agent will be determined by a physician, in view of relevant circumstances of the subject being so treated, including the particular condition (e.g., post-surgical pain), the chosen route of administration, the particular medication being administered in combination with the ALDH-2 inhibitor, the relative activity of the medication, and of course, the age, the weight, the severity of symptoms, the response of the individual subject to the treatment, and the like.

For preparing a solid pharmaceutical composition useful in the methods of the present disclosure, the active ingredient(s) is mixed with a pharmaceutical excipient to form a solid preformulation composition containing a homogeneous mixture of the active ingredient(s) and the excipients. When referring to these preformulation compositions as homogeneous, it is meant that the active ingredient(s) is dispersed evenly throughout the composition so that the composition may be readily subdivided into equally effective unit dosage forms such as tablets, pills and capsules. Tablets or pills may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Pharmaceutical compositions that can be administered by inhalation or insufflation include solutions and suspensions in pharmaceutically acceptable, aqueous organic solvents, or mixtures thereof, and powders. The liquid or solid compositions may contain suitable pharmaceutically acceptable excipients as described herein and as known in the art. In some embodiments, the pharmaceutical composition(s) of the medication and the ALDH-2 inhibitor can be administered by the oral or nasal respiratory route for local or systemic effect. In some embodiments, the pharmaceutical compositions are prepared in pharmaceutically acceptable solvents which can be nebulized by use of inert gases. These nebulized solutions can be inhaled directly from the nebulizing device or the nebulizing device may be attached to a face mask tent, or intermittent positive pressure breathing machine. In some embodiments, the pharmaceutical composition(s) useful in the methods can be in solution, suspension, or powder compositions and can be administered, orally or nasally, from devices that deliver the formulation in an appropriate manner.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### EXAMPLE 1

### Preparation of Compound (1) - 2,6-dichloro-N-[4-(2-oxo-1,2-dihydro-pyridin-4-yl)-benzyl]-benzamide

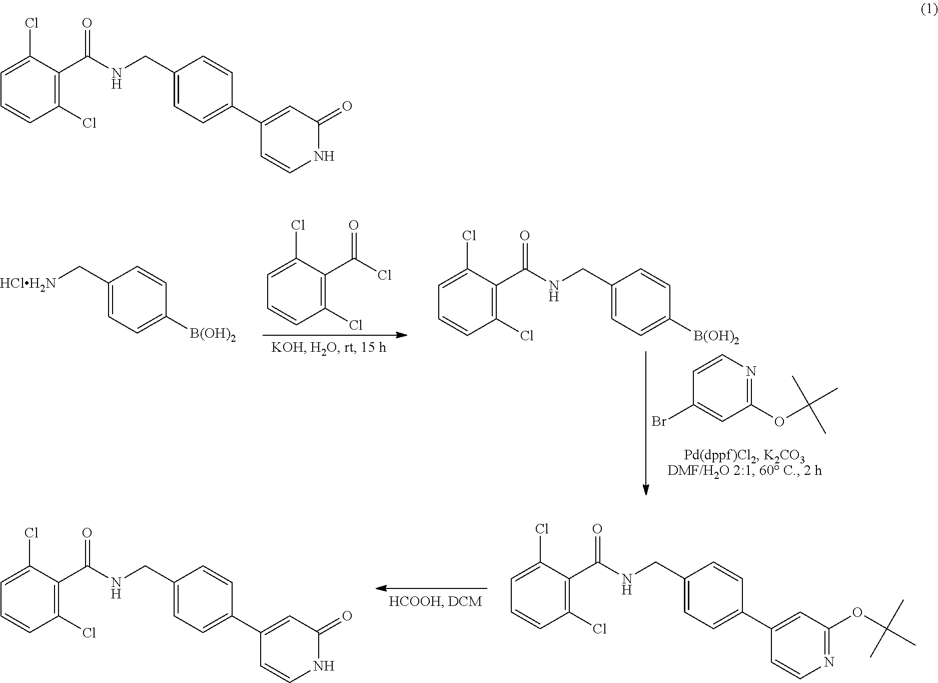

Step 1—Preparation of 4-[(2,6-dichloro-benzoylamino)methyl]phenylboronic acid

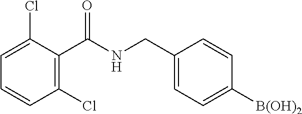 [SMILES: O=C(NCc1ccc(B(O)O)cc1)c1c(Cl)cccc1Cl]

4-(Aminomethyl)phenylboronic acid hydrochloride (5 g, 26.7 mmol) was dissolved in 25 mL water. 16 mL 50% aqueous KOH solution was added followed by 2,6-dichlorobenzoyl chloride (6.7 g, 32 mmol). The mixture was stirred rapidly at room temperature over night. Acidification with 1N HC1 gave a thick, white precipitate which was filtered, washed with water and dried giving 4-[(2,6-dichloro-benzoylamino) methyl]phenylboronic acid as a white powder in quantitative yield.

Step 2—Preparation of N4-[(2-tert-butoxy-pyridin-4-yl)-benzyl]-2,6-dichloro -benzamide

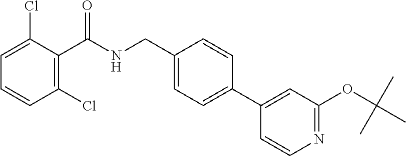 [SMILES: CC(C)(C)Oc1cc(-c2ccc(CNC(=O)c3c(Cl)cccc3Cl)cc2)ccn1]

4-[(2,6-Dichloro-benzoylamino)methyl]phenylboronic acid (5 g, 15.4 mmol), potassium carbonate (5 g), and [1,1′ bis(diphenylphosphino)ferrocene] dichloropalladium (II) (0.56g, 0.77 mmol) were combined in a round bottom flask. 4-Bromo-2-(t-butoxy) pyridine (3.55g, 15.4 mmol) was dissolved in 20 mL DMF and added to the flask under stirring. The flask was flushed with nitrogen and 10 mL water was added. The reaction mixture was stirred at 70° C. for two hours. After cooling the mixture was poured into 300 mL ethyl acetate and washed with water and brine. The organic phase was dried with magnesium sulfate and evaporated under vacuum. The crude N4-[(2-tert-butoxy-pyridin-4-yl)-benzyl]-2,6-dichloro-benzamide was further purified by silica gel chromatography (eluent: hexone/ethyl acetate 1:1).

Step 3—Preparation of 2,6-Dichloro-N-[4-(2-oxo-1,2-dihydro-pyridin-4-yl)-benzyl]-benzamide

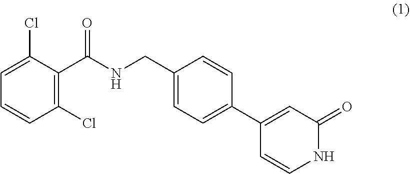 [SMILES: O=C(NCc1ccc(-c2cc[nH]c(=O)c2)cc1)c1c(Cl)cccc1Cl]

N-[4-(2-tert-Butoxy-pyridin-4-yl)-benzyl]-2,6-dichloro-benzamide was dissolved in 30 mL dichloromethone and 12 mL of 98% formic acid. The mixture was stirred at 40° C. for three hours after which the volatile components were evaporated under vacuum. The residue was triturated with ethyl acetate, filtered, washed with ethyl acetate and dried giving 2,6-dichloro-N-[4-(2-oxo-1,2-dihydro-pyridin-4-yl)-benzyl]-benzamide (4.34 g, 75.5% yield over two steps) as white powder. C₁₉H₁₄Cl₂N₂O₂: MS m/z: 373 (MH⁺) ¹H NMR (DMSO-d₆): δ11.56 (s, 1H), δ9.21 (t, J=5.6 Hz, 1H), δ7.67 (d, J=8.0 Hz, 2H), δ7.46 (m, 6H), δ6.57 (d, J=1.2Hz, 1H), δ6.49 (dd, J=6.8 Hz, J′=1.6 Hz, 1H), δ4.50 (d, J=6.0 Hz, 2H.

### EXAMPLE 2

### Preparation of Compound (2)—phosphoric acid mono-(4-{4-[(2,6-dichloro-benzoylamino)-methyl]-phenyl}-2-oxo-2H-pyridin-1-ylmethyl) ester

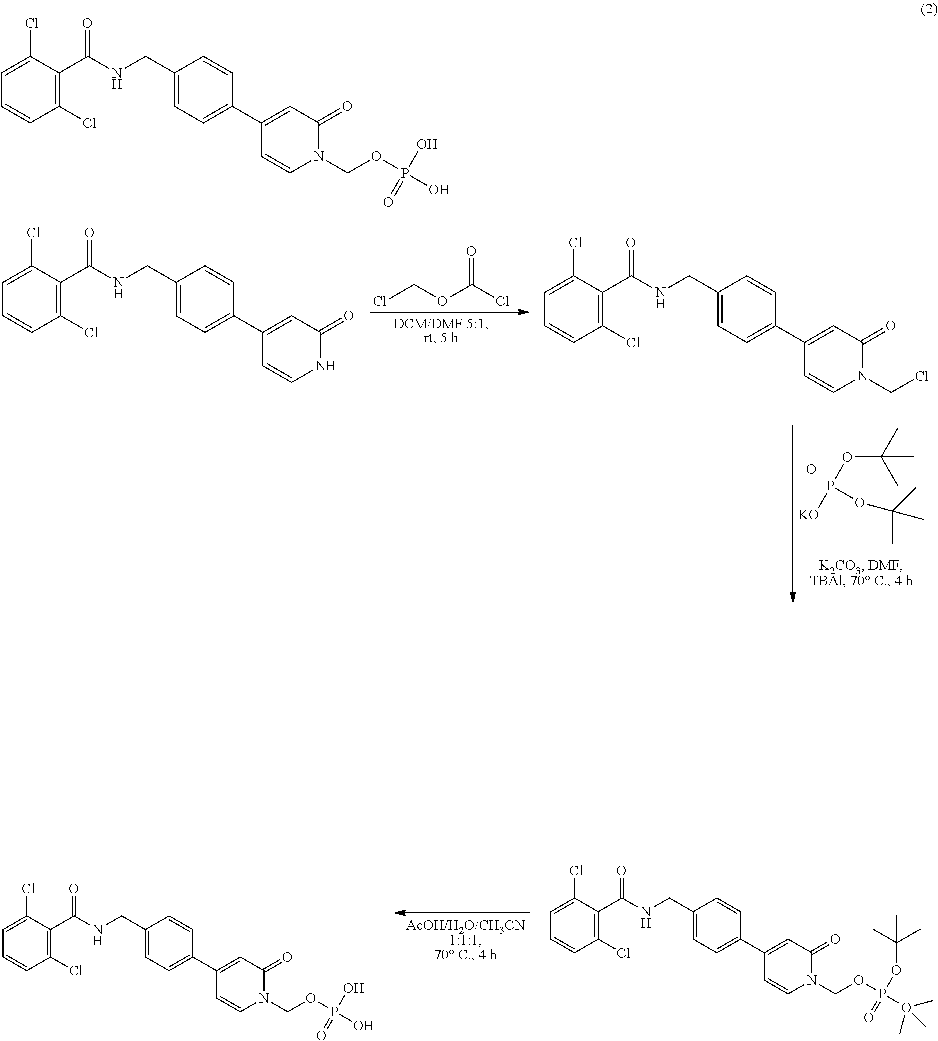

Step 1—Preparation of 2,6-dichloro-N-[4-(1-chloromethyl-2-oxo-1,2-dihydro -pyridin-4-yl)-benzyl]-benzamide

2,6-Dichloro-N-[4-(2-oxo-1,2-dihydro-pyridin-4-yl)-benzyl]-benzamide (1.62 g, 4.34 mmol) (compound (1)), was suspended in 15 mL dichloromethone. Chloromethylchloroformate (0.672 g, 5.21 mmol) was added followed by 3 mL DMF. The mixture was stirred at room temperature for five hours. After diluting with 200 mL ethyl acetate, the organic phase was washed with saturated, aqueous sodium bicarbonate solution and brine, dried with magnesium sulfate and evaporated under vacuum. The crude 2,6-dichloro-N-[4-(1 -chloromethyl-2-oxo-1,2-dihydro-pyridin-4-yl)-benzyl]-benzamide was used in the following step without further purification.

Step 2—Preparation of phosphoric acid di-tert-butyl ester 4-{4-[(2,6-dichloro -benzoylamino)-methyl]-phenyl}-2-oxo-2H-pyridin-1-ylmethyl ester

2,6-Dichloro-N-[4-(1-chloromethyl-2-oxo-1,2-dihydro-pyridin-4-yl)-benzyl] -benzamide from the previous step was dissolved in 50 mL DMF. Potassium carbonate (1 g) was added followed by potassium di(t-butyl)phosphate (2 g) and tetrabutylammonium iodide (50 mg). The mixture was stirred at 70° C. for four hours after which it was poured into 300 mL ethyl acetate. The organic phase was washed with water and brine, dried with magnesium sulfate and evaporated under vacuum. The crude product was further purified by silica gel chromatography (eluent: ethyl acetate), giving phosphoric acid di-tert-butyl ester 4-{4-[(2,6-dichloro-benzoylamino)-methyl]-phenyl}-2-oxo-2H-pyridin-1-ylmethyl ester as a colorless oil which slowly crystallized.

Step 3—Preparation of phosphoric acid mono-(4-[4-[(2,6-dichloro-benzoylamino)-methyl]-phenyl]-2-oxo-2H-pyridin-1-ylmethyl) ester

Phosphoric acid di-tert-butyl ester 4-[4-[(2,6-dichloro-benzoylamino)-methyl]-phenyl]-2-oxo-2H-pyridin-1-ylmethyl ester from the previous step was dissolved in 20 mL acetonitrile, 20 mL acetic acid and 20 mL water, and heated at 70° C. for four hours. All volatile components were evaporated under vacuum and the residue was dissolved in 10 mL DMF. Slow addition of acetonitrile (˜60 mL) precipitated the product which was filtered, washed with more acetonitrile and dried, giving phosphoric acid mono-(4-{4-[(2,6-dichloro-benzoylamino)-methyl]-phenyl}-2-oxo-2H-pyridin-1-ylmethyl) ester (1.17 g, 56% over three steps) as a white powder. ¹H-NMR (DMSO) δ: 9.23 (t, J=6.2 Hz, 1H), 7.73 (d, J=8.4 Hz, 2H), 7.71 (d, J=8.4 Hz, 1H), 7.52-7.40 (m, 5H), 6.72 (d, J=1.6 Hz, 1H), 6.65 (dd, J=7.2 Hz, J=1.6 Hz, 1H), 5.61 (d, J=9.6 Hz, 2H), 4.52 (d, J=6.4 Hz, 2H). MS: 483/485 (MH⁺).

### EXAMPLE 3

### Formulation of Pharmaceutical Compositions

This example illustrates formulations of the pharmaceutical compositions that can be used in the methods of present disclosure for reducing or preventing acquisition of addiction in a patient using a dopamine-producing agent.

Hard gelatin capsules: The ingredients listed below are mixed and filled into hard gelatin capsules:

| | Quantity |
|---|---|
| Ingredient | (mg/capsule) |
| Active Ingredient | 30.0 |
| Starch | 305.0 |
| Magnesium stearate | 5.0 |

240 mg Tablets: The ingredients listed below are blended and compressed to form 240 mg tablets:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

120 mg Tablets: The ingredients listed below are blended and compressed as described below to form 120 mg tablets:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch, and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50 ° C. to 60 ° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Suppositories: Suppositories each containing 25 mg of active ingredient, are made as follows:

| Ingredient | Quantity |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions: A suspension containing 50 mg of active ingredient per 5.0 mL dose, is made as follows:

| Ingredient | Amount |
|---|---|
| Active Ingredient | 50.0 mg |
| Xanthan gum | 4.0 mg |
| Sodium carboxymethyl cellulose (11%) | |
| Microcrystalline cellulose (89%) | 50.0 mg |
| Sucrose | 1.75 g |
| Sodium benzoate | 10.0 mg |
| Flavor and Color | q.v. |
| Purified water to | 5.0 mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Subcutaneous: a subcutaneous formulation is prepared as follows:

| Ingredient | Quantity |
|---|---|
| Active Ingredient | 5.0 mg |
| Corn Oil | 1.0 mL |

Injectable: an injectable formulation is prepared by combining the following ingredients:

| Ingredient | Quantity | |
|---|---|---|
| Active ingredient | 2.0 | mg/mL |
| Mannitol, USP | 50 | mg/mL |
| Gluconic acid, USP | q.s. (pH 5-6) | |
| Water (distilled, sterile) | q.s. to 1.0 | mL |

| | |
|---|---|
| Nitrogen Gas, NF | q.s. |

Topical: a topical preparation is prepared by combining the following ingredients as described below:

| Ingredients | Quantity (g) |
|---|---|
| Active ingredient | 0.01-1 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

EXAMPLE 4: Combination Therapy to Prevent Acquisition of Conditioned Response Self-Administration Behavior for Opioid Medication

This example illustrates an experimental study for determining the dose-effect function of the ALDH-2 inhibitor of compound (2), on the acquisition of conditioned response self-administration behavior in rats for an opioid medication (e.g. remifentanil hydrochloride).

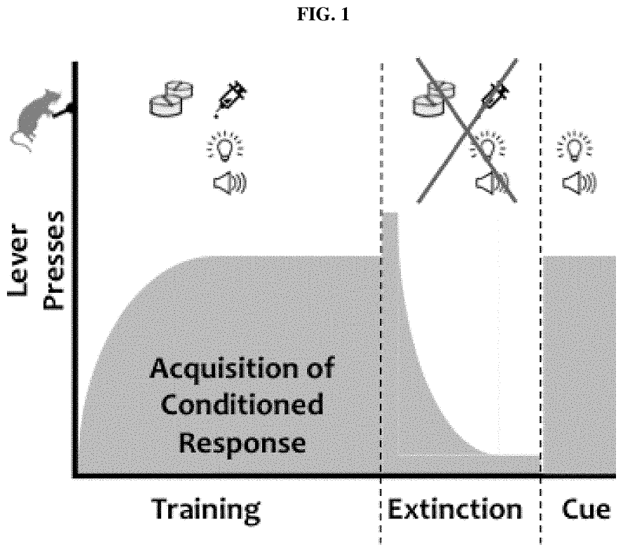

Experimental Design and Protocol: The general design of the study is illustrated schematically in FIG. 1. First, rats receive oral doses of the ALDH-2 inhibitor of compound (2) or vehicle during 10 consecutive days of self-administration training for the dopamine-producing agent, the opioid remifentanil hydrochloride and the number of lever presses are measured. They also receive a light and a sound cue with opioid self-administration. After, the rats may experience one week of forced abstinence from the opioid self-administration during which the rats do not receive opioid, the light or sound cues, or the ALDH-2 inhibitor of compound (2). This abstinence period models attempts in humans to cease opioid self-medication. Following, this one-week abstinence period, rats are re-exposed to the light and sound cues only (with no opioid administration), and the number of lever pressings may also be measured.

Subjects: Adult male Sprague-Dawley rats are singly housed under standard laboratory conditions in a vivarium facility next to a testing room to minimize stress induced by transport. The day-night cycle is reversed cycle so that the rats are in their active phase during behavioral testing. All rats have ad lib access to water and are fed the same type of rat chow once daily so as to maintain approximately 85% ad lib weight with food amounts adjusted from 8-16 g per day to provide a lean healthy growth curve. Twelve rats are used per dose (total of N=48 rats).

Preparation of ALDH-2 Inhibitor: The ALDH-2 inhibitor compound, compound (2), is prepared as described elsewhere herein. Solutions of compound (2) are prepared in pyrogen-free glassware in water while monitoring the pH which was adjusted to 7.8 to 8.0 with 5 N NaOH. The solution is administered to the rats by oral gavage in a volume of 5 mL/kg BW.

Preparation of Opioid Medication: Solutions of remifentanil hydrochloride are prepared in pyrogen-free glassware in sterilized isotonic saline. The pH of the solutions is adjusted to 7.0 using NaOH and then the solutions passed through a 0.2 μm filter (Millipore Corp, Billerica, Mass., USA). All solutions are kept refrigerated in the dark between experiments. All rats are also administered 5 mL/kg saline

Behaviorial Training: For behavioral training, rats are placed in dual lever operant test chambers (Med Associates, Georgia, VT, USA). Each chamber is equipped with a tone generator, house light, cue light above each lever, and a metal tether to cover the drug delivery line. The drug delivery line is made of polyethylene tubing with huber needles for access to ports and catheters. Each catheter is connected to a Micro Liter Syringe Pump. During each session, the rats wear infusion harnesses to connect them to the tethers. A computer programmed with MED-PC software is used to control experimental events and data collection. Initially, rats are trained daily to press the levers for food pellet reinforcers with tutor sessions lasting 30 minutes. The training procedure is carried out just before the surgery for catheter implantation, which is just before the onset of remifentanil access. Half of the animals are rewarded for responding on the right lever and half for responding on the left. Only the cue light over the correct lever is illuminated while the light over the incorrect lever remains off. Responses on the correct lever are rewarded by immediate delivery of one 45-mg food pellet and activation of the feedback tone for 0.5 seconds and light illumination. There are no timeouts in the tutor sessions.

Catheter Implantation: After the food pellet behavioral training sessions, chronically indwelling intravenous jugular catheters are surgically implanted i.v. under ketamine anesthesia. A plastic SoloPort is attached intraoperatively to a polyurethane catheter and inserted into a subcutaneous interscapular pocket and sutured to underlying fascia. The catheters provide access for remifentanil self-administration by i.v. infusion. The catheters are flushed daily with a 0.3 mL solution containing 100 U/mL heparinized saline. After self-administration testing sessions, the remifentanil remaining in each port is drawn out and a sterile lock is infused, consisting of heparinized saline 500 U/mL with 0.4 mg Gentamicin as an antibiotic. Barbituate injection tests through the catheter are used to verify patency (see, Rezvani et al., “Effects of sazetidine-A, a selective α4β2 nicotinic acetylcholine receptor desensitizing agent on alcohol and nicotine self-administration in selectively bred alcohol-preferring (P) rats,” Psychopharmacology (2010) 211 2: 161-174; and Levin et al., “Sazetidine-A, a selective α4β2 nicotinic receptor desensitizing agent and partial agonist, reduces nicotine self-administration in rats,” Journal of Pharmacology and Experimental Therapeutics (2010), 332 3: 933-939).

Remifentanil Self-Administration Training: Two to four days following surgical implantation, the rats begin opioid self-administration training sessions with remifentanil hydrochloride solution self-administered i.v. as the reinforcer. Two hours before each remifentanil self-administration training session, one of the three dosages of the ALDH-2 inhibitor compound (2) to be tested (9, 18, or 36 mg/kg) or the same volume of the vehicle, is administered orally. The benchmark infusion dose of the remifentanil solution is 0.9 μg/kg/infusion. FR is set at FR-1, and each remifentanil infusion self-administration training session is 1-hour (see, Levin et al., “Reduction of nicotine self-administration by chronic nicotine infusion with H1 histamine blockade in female rats,” Psychopharmacology (2016) vol. 233: 3009-3015; and Rezvani et al., “Acute oral 18-methoxycoronaridine (18-MC) decreases both alcohol intake and IV nicotine self-administration in rats,” Pharmacology Biochemistry and Behavior (2016), vol. 150-151: 153-157). During the sessions, a lever press on the active side results in the activation of the feedback tone for 0.5 second, and the immediate delivery of one 50-μl infusion of remifentanil in less than 1 second. Each infusion is immediately followed by a one-minute timeout in which the house light goes on, cue lights go out, and responses are recorded but not reinforced. The acquisition of the conditioned response is measured by the number of lever presses that results in remifentanil self-administration (“infusions”). The testing is carried out for 10 consecutive days.

Abstinence Period and Re-exposure to Cues: Following the remifentanil self-administration training period there is a one-week period of forced abstinence from remifentanil self-administration. The administration of compound (2) is also halted during this period. Following, this one-week abstinence period, the rats are re-exposed to the cue light and tone but without any remifentanil or compound (2) administration, and the number of lever pressings is measured.

Statistical Analysis: Data are evaluated with analysis of variance (ANOVA) with an analysis for within- and between-subject factors. Alpha of p<0.05 (two-tailed) are used as the threshold for statistical significance. The N=12/dose has been found in previous studies to provide sufficient power to detect biologically significant effects.

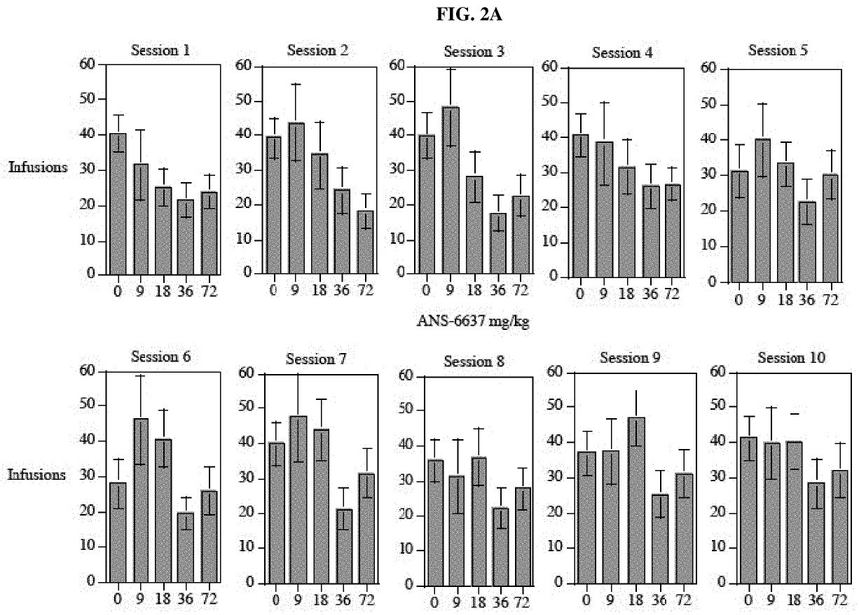

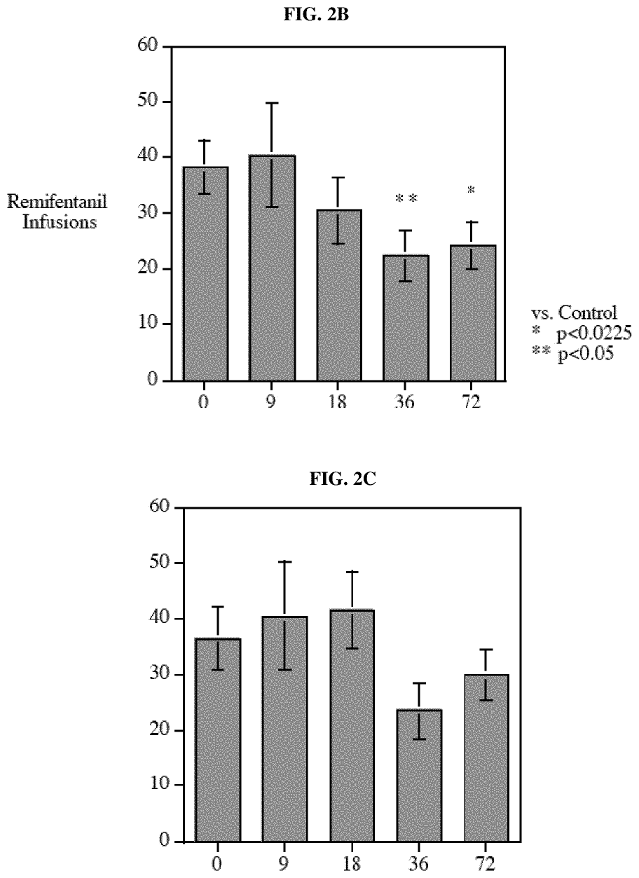

Results: The results shown in FIG. 2A are the mean number of lever presses (infusions) by the group of rats receiving vehicle (0), 9, 18, 36, and 72 mg compound (2) each day of the 10-day period of acquisition of the conditioned response. FIG. 2B and FIG. 2C depict the responses observed on days 1-5 and 6-10, respectively. Oral doses of 36 and 72 mg/kg of compound (2) administered to rats during the remifentanil self-administration training period resulted in statistically significant decreases in lever presses during days 1-5 but not 6-10. No difference was observed between groups upon cue-induced lever pressing (data not shown).

This study shows that administration of an ALDH-2 inhibitor to rats during remifentanil self-administration training can reduce self-administration to delay the onset of the conditioned response. This study thus demonstrates that the ALDH-2 inhibitor of compound (2) exhibits a positive dose response in preventing the acquisition of conditioned response behavior in a rat model for opioid self-medication cessation in humans.

### EXAMPLE 5

### Randomized Double-Blind Placebo-Controlled Parallel Dose Design Study of an ALDH-2 Inhibitor-Opioid Combination Therapy to Reduce Post-Surgical Opioid Consumption and the Incidence of DSM5 Opioid Use Disorder in 200 Patients Undergoing Total Hip or Total Knee Replacement (THR/TKR) Surgery

This example illustrates a study of the extent to which the ALDH-2 inhibitor, compound (2), when administered in combination with an opioid medication in human patients following total hip or knee replacement surgery reduces opioid consumption and the acquisition of opioid related conditioned response of craving, drug seeking, and relapse.

Key inclusion criteria: (a) Undergoing total hip or total knee replacement THR/TKR) surgery; (b) age, between 21 and 60; (c) plus, other “standard” inclusion criteria,

Key exclusion criteria: (a) Opioid use in the 3 months prior to surgery; (b) plus, other “standard” exclusion criteria,

General Study Design:
a) 200 patients are enrolled to receive one of three doses of the study drug, compound (2): Placebo, Low, High.
b) Patients begin taking the study drug as soon as possible after surgery - if possible on the day of surgery (day 1) or the day after surgery (day 2) and continue taking the study drug through 3 months post-surgery.
c) Patients have study related visits once prior to surgery and then on days 1 (or 2), 7, 28, 56 and 91.
d) Physicians write an initial post-surgery prescription of an opioid medication for pain as necessary (based on standardized prescription choices) to get to the day 7 visit.
e) Refills are provided, as requested by the patients, at day 7, and thereafter during the protocol. The duration and quantity of refills are standardized based on pending discussions with THR/TKR surgeons.

Study endpoints/assessments:
a) Pills of opioid medication consumed (Primary)
b) Morphine milligram equivalents (MMEs) of opioid consumed
c) Patient reported date of cessation of opioid
d) Earliest date of opioid free urine test
e) # of opioid medication prescriptions filled
f) # of high MME days (high MME day tbd)
g) % patients who are opioid free at day 7, 28, 56 and 91
h) % patients who meet DSM5 Criteria for Opioid Use Disorder at ay 28, 56 and 91.
i) Opioid craving assessment

Study drug supply: The dosage of study drug, compound (2), is administered to the patients as a once-a-day (QD) dose of 100 mg capsules (or matching placebo) packaged in 30 count bottles. Each dose can include as many as 3 capsules.

Results: This study can show the extent to which the study drug, compound (2), when co-administered to human patients with an opioid medication following total hip or knee replacement surgery, can reduce the patient's opioid consumption and the acquisition of an opioid-related conditioned response of craving, drug seeking, and relapse.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the inventions.

What is claimed is:

1. A method of reducing or preventing addiction to a substance that produces a conditioned response in a mammal, the method comprising administering to the mammal a therapeutically effective amount of an ALDH-2 inhibitor in combination with the substance, wherein prior to administering the mammal has not acquired a conditioned response to the substance.

2. The method of claim 1, wherein prior to administering the ALDH-2 inhibitor in combination with the substance, the mammal has not used the substance, been treated with the substance, or otherwise ingested the substance, for a period of time of at least 1 month, at least 3 months, at least 6 months, at least 1 year, or ever.

3. The method of claim 1, wherein the ALDH-2 inhibitor and the substance are:
(i) administered separately and not at the same time;
(ii) administered separately and at the same time;
(iii) administered in a combination dosage form; or (iv) self administered.

4. The method of claim 1, wherein the mammal is a human.

5. The method of claim 1, wherein the substance is a medication, an extract, a food, alcohol, nicotine, amphetamine, or other drug of addiction.

6. The method of claim 5, wherein the substance is a medication; optionally, an opioid medication; which is optionally selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

7. The method of claim 6, wherein the mammal suffers from chronic pain and the medication is an opioid medication, or wherein the mammal has undergone a surgical procedure and the medication is a post-surgical treatment.

8. The method of claim 1, wherein the substance is a medication which comprises a dopamine-producing agent and the mammal is in need of said medication, and wherein prior to the administering of the ALDH-2 inhibitor in combination with the medication the mammal has not acquired a conditioned response to the dopamine-producing agent

9. The method of claim 8, wherein the ALDH-2 inhibitor and the medication are administered in a combination dosage form which optionally comprises a pharmaceutical composition of the ALDH-2 inhibitor, the medication, and a pharmaceutically acceptable carrier, and/or which is optionally formulated in a unit dosage which is optionally an oral unit dosage.

10. The method of claim 1, wherein the ALDH-2 inhibitor is a compound of Formula (I) 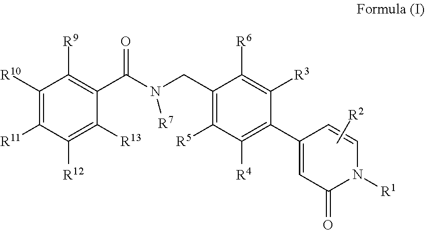
wherein:
R¹ is hydrogen, optionally substituted C₁₋₆ alkyl, —CH₂OH, —CH₂OP(O)(OR²⁰)(OR²¹);
R² is hydrogen, optionally substituted C₁₋₆ alkyl, cycloalkyl, or halo;
each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O-(C₁to C₆-alkyl)-O-(C₁ to C₆-alkyl), cyano, halo, —SO₂NR²⁴R²⁵; or —NR²⁴R²⁵;
R⁷ is hydrogen or optionally substituted C₁₋₆ alkyl;
each of R²⁰ and R²¹ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and
each of R²⁴ and R²⁵ is independently chosen from hydrogen or C₁₋₆ alkyl or when combined together with the nitrogen to which they are attached form a heterocycle; or a pharmaceutically acceptable salt, ester, single stereoisomer, mixture of stereoisomers, or a tautomer thereof.

11. The method of claim 10, wherein
R¹ is hydrogen, methyl, or —CH₂OP(O)(OR²⁰)(OR²¹);
R² is hydrogen, methyl, or fluoro;
each of R³ or R⁴ is independently hydrogen or methyl;
each of R⁵ and R⁶ is independently hydrogen or fluoro;
R⁷ is hydrogen;
R⁹ is hydrogen, chloro, fluoro, or methyl;
R¹⁰ is hydrogen or fluoro;
R¹¹ is hydrogen or —OCH₂CH₂OCH₃;
R¹² is hydrogen or fluoro;
R¹³ is hydrogen, chloro, fluoro, or methyl; and
each of R²⁰ and R²¹ is independently Na⁺, Li⁺, K⁺, or hydrogen; or wherein the compound of Formula (I) is selected from:
2,6-dichloro-N-[4-(2-oxo-1,2-dihydro-pyridin-4-yl)-benzyl]-benzamide;
phosphoric acid mono-(4-[4-[(2,6-dichloro-benzoylamino)-methyl]-phenyl]-2-oxo-2H -pyridin-1-ylmethyl) ester;
2,6-dichloro-4-(2-methoxyethoxy)-N-(4-(2-oxo-1,2-dihydropyridin-4-yl) benzyl)benzamide;
2-chloro-3-fluoro-N-(4-(2-oxo-1,2-dihydropyridin-4-yl)benzyl)benzamide;
2-chloro-6-methyl-N-(4-(2-oxo-1,2-dihydropyridin-4-yl)benzyl)benzamide;
2,6-dimethyl-N-(4-(2-oxo-1,2-dihydropyridin-4-yl)benzyl)benzamide;
2,6-dichloro-N-[4-(6-methyl-2-oxo-1,2-dihydro-pyridin-4-yl)-benzyl]-benzamide;
2-chloro-3,6-difluoro-N-(4-(2-oxo-1,2-dihydropyridin-4-yl)benzyl)benzamide;
2,6-dichloro-N-(3-methyl-4-(2-oxo-1,2-dihydropyridin-4-yl)benzyl)benzamide);
2,6-dichloro-N-(4-(1-methyl-2-oxo-1,2-dihydropyridin-4-yl)benzyl)benzamide;
2,6-difluoro-N-(4-(2-oxo-1,2-dihydropyridin-4-yl)benzyl)benzamide;
2-chloro-6-fluoro-N-(4-(2-oxo-1,2-dihydropyridin-4-yl)benzyl)benzamide;
2,6-dichloro-N-(2-fluoro-4-(2-oxo-1,2-dihydropyridin-4-yl)benzyl)benzamide;
2,6-dichloro-N-(4-(5-fluoro-2-oxo-1,2-dihydropyridin-4-yl)benzyl)benzamide;
2,6-dimethyl-N-(4-(2-oxopiperidin-4-yl)benzyl)benzamide;
or a pharmaceutically acceptable salt, single stereoisomer, mixture of stereoisomers, or a tautomer thereof.

12. The method of claim 10, wherein the compound of Formula (I) is compound (1): 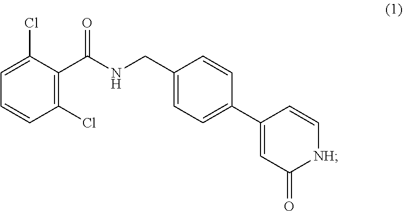 [SMILES: O=C(NCc1ccc(-c2cc[nH]c(=O)c2)cc1)c1c(Cl)cccc1Cl]
or a pharmaceutically acceptable salt, or a tautomer thereof.

13. The method of claim 10, wherein the compound of Formula (I) is compound (2): 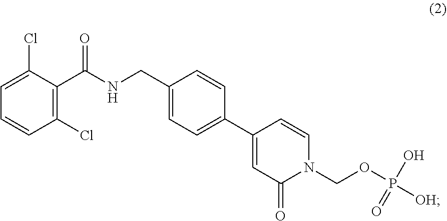 [SMILES: O=C(NCc1ccc(-c2ccn(COP(=O)(O)O)c(=O)c2)cc1)c1c(Cl)cccc1Cl]
or a pharmaceutically acceptable salt, ester, or a tautomer thereof.

14. The method of claim 1, wherein the ALDH-2 inhibitor is a compound comprising an isoflavone structure.

15. A pharmaceutical composition comprising a therapeutically effective amount of an ALDH-2 inhibitor, a substance comprising a dopamine-producing agent that produces a conditioned response in a mammal, and a pharmaceutically acceptable carrier.

16. The pharmaceutical composition of claim 15, wherein the substance is a medication, an extract, a food, alcohol, nicotine, amphetamine, or other drug of addiction.

17. The pharmaceutical composition of claim 16, wherein the substance is an opioid medication;
optionally, wherein the opioid medication selected from the group consisting of alfentanil, buprenorphine, butorphanol, carfentanil, codeine, dipanone, fentanyl, hydrocodone, hydromorphone, oxycodone, oxymorphone, levorphanol, lofentanil, morphine, meperidine, methadone, remifentanil, heroin, tramadol, etorphine, dihydroetorphine, sufentanil, and the stereoisomers, polymorphs, metabolites, prodrugs, pharmaceutically acceptable salts, and mixtures thereof.

18. The pharmaceutical composition of claim 15, wherein the ALDH-2 inhibitor is a compound of Formula (I) is 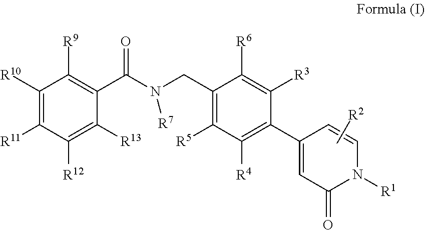
wherein:
R¹ is hydrogen, optionally substituted C₁₋₆ alkyl, —CH₂OH, —CH₂OP(O)(OR²⁰)(OR²¹);
R² is hydrogen, optionally substituted C₁₋₆ alkyl, cycloalkyl, or halo;
each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O-(C₁ to C₆-alkyl)-O-(C₁ to C₆-alkyl), cyano, halo, —SO₂NR²⁴R²⁵; or —NR²⁴R²⁵;
R⁷ is hydrogen or optionally substituted C₁₋₆ alkyl;
each of R²⁰ and R²⁴ is independently Na⁺, Li⁺, K⁺, hydrogen, C₁₋₆ alkyl; or R²⁰ and R²⁴ can be combined to represent a single divalent cation Zn²⁺, Ca²⁺, or Mg²⁺; and
each of R²⁴ and R²⁵ is independently chosen from hydrogen or C₁₋₆ alkyl or when combined together with the nitrogen to which they are attached form a heterocycle; or a pharmaceutically acceptable salt, ester, single stereoisomer, mixture of stereoisomers, or a tautomer thereof.

19. The pharmaceutical composition of claim 18, wherein the compound of Formula (I) is compound (1): 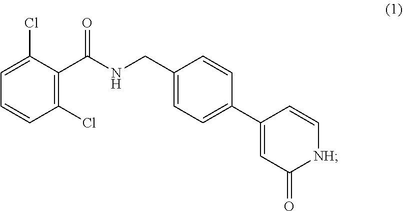 [SMILES: O=C(NCc1ccc(-c2cc[nH]c(=O)c2)cc1)c1c(Cl)cccc1Cl]
or a pharmaceutically acceptable salt, or a tautomer thereof.

20. The pharmaceutical composition of claim 18, wherein the compound of Formula (I) is compound (2): 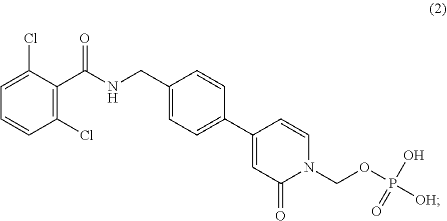 [SMILES: O=C(NCc1ccc(-c2ccn(COP(=O)(O)O)c(=O)c2)cc1)c1c(Cl)cccc1Cl]
or a pharmaceutically acceptable salt, ester, or a tautomer thereof.
